(19) United States
(12) Patent Application Publication
(10) Pub. No.: US 2007/0185346 A1
(43) Pub. Date: Aug. 9, 2007

(54) Kit for automated resolving agent selection and method thereof

(76) Inventor: Niteen A. Vaidya, Cupertino, CA (US)

Correspondence Address: PETERS VERNY , L.L.P., 425 SHERMAN AVENUE, SUITE 230, PALO ALTO, CA, 94306, US

(21) Appl. No.: 11/347,532

(22) Filed: Feb. 3, 2006

(51) Int. Cl. C07C 229/02

(52) U.S. Cl. 562/401

(57) ABSTRACT

The present invention concerns an improved method and a tray or kit, which is useful to select quickly the optimum resolution agents, combinations and conditions to separate optical isomers. The tray of 24, 48, 96 or more samples is examined simultaneously visually or by standard analytical techniques.

### BACKGROUND OF THE INVENTION

1. Field of the Invention

The present invention concerns an improved tray (or kit), which is useful to select quickly the optimum resolving agents and solvents, combinations and conditions to separate optical isomers. The tray of 24, 48 or more samples can be examined visually or by standard analytical techniques.

2. Description of Related Art

In the pharmaceutical sciences, it has long been known that useful organic compounds are isomeric. Often one isomer is therapeutic, while the other isomer has a neutral health benefit or more likely has significant harmful side effects. This was the case in the 1960s with the drug, thalidamide. One isomer induced the desired sleep, but the other isomer was teratogenic, causing significant defects in the in utero fetus by reducing blood vessel growth. The thalidamide isomer that causes reduction in blood vessel growth is now being examined in cancer therapy to reduce blood vessel growth to tumors.

Similarly, the anti-arthritis drug, naproxen—the active ingredient in the anti-inflammatory ALEVE® (Proctor & Gamble, Cincinnati, Ohio)—is an optically active isomer. One isomer causes liver dysfunction; the other isomer is therapeutic for arthritis. Naproxen has strict controls and limits on the active isomer released for public use.

A major limitation to resolution of mixtures of racematic compounds concerns the identification of optimal diastereoisometric salt crystallization conditions. This screening often takes too long.

### BACKGROUND FOR CHIRAL KITS FOR SCREENING RESOLVING AGENTS

Chiral technology has wide application in specialty chemicals such as pharmaceuticals, herbicides, pheromones, liquid crystals, non-linear optical materials and polymers, aroma and flavors, vitamins, sweeteners, dyes and pigments, etc. The worldwide market for chiral products is over $200 billion. In the pharmaceutical area alone it is $115 billion for single enantiomer drugs. There are three basic approaches in getting enantiopure compounds: 1) chiral synthesis, 2) separation of racemic mixtures, and 3) enzymatic degradation of one enantiomer. There are several clear advantages of separation of racemates over that chiral synthesis. First, they tend to be simpler processes. Second, they often give much better volumetric productivities for equivalent optical purities. Third and perhaps most importantly, the desired enantiomeric excess (ee) is achieved by adjusting the level of conversion (in the case of diastereomeric crystallization if one increases the number of recrytallizations the (ee) is subsequently increased at each stage).

Examination of a representative group of such drugs shows that roughly 65% have optical activity after classical resolution (diastereomeric crystallization). There are clearly many instances where resolution is both economically viable and the preferred method. The main challenges involved with this method are to select optimum resolving agent/nature and composition of the solvent within a given time frame (pre-manufacturing decision). This selection is often time consuming, tedious, and labor intensive. Recognizing this as a unique commercial opportunity where today no other comparative product is available in the market, chiral kits (96, 192, 384, etc.) vials for easy robotic manipulation and step-by-step instructions for simple experimental set up are available. A series of tests on racemates to choose the combination of resolving agents and solvents may be done in parallel to maximize the chances of success. The protocol of resolution experiments is developed so that considerable time is saved before the pre-manufacturing decision is made on the choice of resolving agent/solvent and conditions of resolution.

Chiral technology has wide application in specialty chemicals such as pharmaceuticals, herbicides, pheromones, liquid crystals, non-linear optical materials and polymers, aroma and flavors, vitamins, sweeteners, dyes and pigments, etc. The worldwide market for chiral products is over $200 billion. In the pharmaceutical section alone it is $115 billion for single enantiomers drugs (Ref. 1). It has been recognized for a long time that the shape of a molecule has considerable influence on its physiological properties. Differentiation within enantiomer pairs are numerous and often dramatic. A few examples are given below which emphasize the reasons for commercial interest and incentive for producing optically pure materials by methods applicable to at least multigram amounts and in many cases to hundreds or thousands of tons.

Examples of fine chemicals, which show the effect of chirality (Table 1)

TABLE 1
CHIRAL EFFECT ON PROPERTIES

| | | |
|---|---|---|
| Compound | Isomer | Effect |

Pharmaceuticals

| | | |
|---|---|---|
| Thalidomide | S-Isomer | Teratogenic |
| | R-Isomer | Sleep inducing |
| Barbiturates | S-Isomer | Depressant |
| | R-Isomer | Convulsant |
| Opiates | R,S-Isomer | Narcotics |
| | S,R-Isomer | Non-addictive cough-mixture |
| Labetalol | R,S-Isomer | Alpha-blocker |
| | S,R-Isomer | Beta-blocker |
| Penicillamine | D-Isomer | Anti-arthritic |
| | L-Isomer | Toxic |

Food/Flavor

| | | |
|---|---|---|
| Aspartame | R,R-Isomer | Sweet taste |
| | S,R-Isomer | Bitter taste |
| Carvone | R-Isomer | Spearmint odor |
| | S-Isomer | Caraway odor |
| Limonene | R-Isomer | Orange odor |

Vitamins

| | | |
|---|---|---|
| Ascorbic acid | L-Isomer | Antiscorbutic acid |

Insecticide

| | | |
|---|---|---|
| Bermethrin | d-Isomer | More toxic than I-Isomer |

Herbicide

| | | |
|---|---|---|
| Fluazifop butyl | R-Isomer | Plant growth regulator |
| Paclobutrazol | R-Isomer | Fungicide |
| | S-Isomer | Plant growth regulator |

There are two basic approaches in obtaining chiral compounds: asymmetric synthesis and resolution.

Asymmetric synthesis often requires auxiliary chiral synthesis or asymmetric catalysis. Resolution involves separation methods such as chromatography, polymer-supported liquid membrane and preferential or diastereometric crystallization. Asymmetric synthesis should be, in principle, the most cost-effective method for producing single-enantiomer products, because all the precursors are converted to the desired enantiomer. However, in industry the decision to implement an asymmetric synthesis approach is typically based on an assessment of efficiency and cost. Among the factors considered are (1) the catalyst efficiency (that is, the number of product molecules produced per molecule of the catalyst); (2) the availability of the metal, the ligand, and the starting materials (especially critical for low value products); and (3) reaction conditions, such as very low temperature or high pressure, and reaction kinetics.

Chiral chromatography is a useful technique for small-scale resolution of racemic mixtures (less than one kilo of material). Several ways to obtain optically pure material, such as asymmetric synthesis (introduce chirality during synthetic sequence), synthesis using chiral pool and stereoselective synthesis using enzymes or chemicals and resolution of racemic mixture using either chromatography or liquid membrane or chemical resolution.

Diastereometric crystallization is widely used in the separation of racemic mixtures even though the theoretical yield is only 50%. But if unwanted isomer is racemized back to the mixture, which sets-up a recycle process to yield the desired optical isomer, which would have an unprecedented economic advantage over other methods².

Utilizing phase diagrams generally speeds up the selection of a good resolving agent and determination of the best crystallization conditions. However, the selection process is still very empirical and trial and error is the best solution to the criteria.

An alternative to diastereomeric salt formation is direct, preferential crystallization of the desired enantiomer, usually initiated by seeding with a pure enantiomer. If applicable, preferential crystallization of enantiomers is a highly economic approach, and Merck, for example, has used it with the great success in the manufacture of alpha-methyl-DOPA (alpha-methyl-L-dihydroxyphenylalanine). However, in practice the method has limited application because it can be applied only to a conglomerate, i.e. a mechanical mixture of crystals of the two enantiomers. In contrast, a true racemic compound where both enantiomers exist in a unit cell cannot be resolved by preferential crystallization. Unfortunately, less than 20% of all known racemates are conglomerates and therefore the remainder are true racemic compounds and cannot be separated by preferential crystallization. Differential scanning calorimeter to obtain a melting point diagram is one method used to assign to which of two classes a racemate belongs.

TABLE 2
OPTICALY ACTIVE PHARMACEUTICALS PRODUCED
(WHOLLY OR PARTIALLY)
USING CRYSTALLIZATION TECHNIQUE
Worldwide Sales

| | Therapeutic | | |
|---|---|---|---|
| Product | Class | Resolving Agent | ($Millions) |
| Amoxycillin | Antibiotics | | 2000 |
| Ampicillin | Antibiotics | (D-Camphor- | 1800 |
| | | sulphonic acid) | |
| Captopril | Cardiovascular | | 1580 |
| Diltiazem | Calcium | (+)- | 980 |
| | antagonist | Phenethylamine | |
| Naproxen | Anti- | (−) | 971 |
| | inflammatory | (Cinchonidine) | |
| Cefalexin | Antiiotic | | 900 |
| Timolol | Cardiovascular | | 325 |
| Cefadroxil | Antibiotic | | 300 |
| ∞-Methyldopa | Cardiovascular | | 225 |
| Chloro- | Antibiotic | (D-Camphor- | 80 |
| ampheicol | | sulphonic acid) | |
| Dextro- | Antitussive | | 50 |
| methorphan | | | |
| Ethambutol | Tuberculostatic | (L-Tartaric | 50 |
| | | acid) | |

(See Chirotechnology, by R. Sheldon, ed, Marcel Dekker, London, 1992)

Examination of a representative group of such drugs shows that roughly 65% owe there optically activity to classical resolution. There are clearly many instances where resolution is both economically viable and preferred method.

Diastereometric crystallization has the advantage of relative simplicity and requires only standard production equipment. From the practical point of view, the method is flexible and suited intermittent batch production, which is often the practice in pharmaceutical manufacture. While the occurrence of desirable crystal behavior and solubilities are in large measure unpredictable, a systematic search for exploitable properties at all relevant points in a sequence will reward the effort and should be part of the modus operandi of the process development chemist. For example, if a substance is readily racemized and a crystallization-induced asymmetric transformation (deracemization) is possible, it offers an extremely attractive industrial option.

There are two types of diastereomers: (1) ionic/salt; and (2) covalent/neutral. Covalent diastereomers are easier to separate by HPLC than are ionic diastereomers. Even so, covalent diastereomers are not preferred because their formation is not as easy as that of salt; nor is their decomposition. Moreover, the forward and reverse reactions are more subject to racemization of chiral centers than is salt formation.

The screening of resolving agents and optimization of resolution:

The initial problem associated with diastereomeric crystallization is to choose the right resolving agent and the nature and composition of the solvent. This can be time consuming, tedious, and labor intensive. Few important points one must take into consideration are:
1. The diastereomeric salt must crystallize well and there must be an appreciable difference in solubility between two salts.
2. The complex between the resolving agent and the substance to be resolved should be easily formed, and the resolving agent should be easily recoverable in a pure state from the salt following the crystallization step.
3. In general, a resolving agent should be available in an optically pure form because a substance to be resolved cannot be obtained in a higher state of optical purity than their resolving agent by mere crystallization of diastereoisomers.
4. The chiral center should be as close as possible to the functional group responsible for salt formation.
5. An agent must be chemically stable and not racemize under the conditions of the resolution process.
6. Resolving agent should be available as both enantiomers so that both forms of the substrate can be prepared.
7. For industrial purposes, a resolving agent should be relatively inexpensive and readily recoverable in high yield after completion of the resolution.

There are no empirical rules that one of skilled in the art can adhere to when it comes to choosing the optimum resolving agent and solvent combination. Fortunately, the number of commercial quantity resolving agents is limited and one can devise standard protocol to screen resolving agents with that of solvents. The table below provides some common resolving agents:

Examples of fine chemicals, which show the effect of chirality (table 1)

TABLE 3
COMMONLY USED RESOLVING AGENTS

| Acids | Bases |
|---|---|
| Tartaric acid (+)(−) | ∞-Methylbenzylamine (+)(−) |
| Dibenzoyltartaric acid (+)(−) | Ephedrine (+)(−) |
| Mandelic acid (+)(−) | 2-Amino-1-butanol (+)(−) |
| Camphoric acid (−) | Quinine (−) |
| Malic acid (+)(−) | Quinidine (+) |
| 1-Camphor-10-Sulphonic acid (+)(−) | Cinchonidine (−) |
| Pyroglutamic acid (+)(−) | Cinchonine (+) |
| ∞-Methoxyphenylacetic acid (+)(−) | Brucine (−) |
| ∞-Methoxy-∞-trifluoromethylphenyl | Dehydroabietylamine (+) |
| Acetic acid (+)(−) | |

For important commercial applications some times a company designs their own resolving agents, such as chiral phosphoric acid developed by andeno and citramalic acid by Lonza. Syntex developed n-methyl-D-glucamine (prepared from D-glucose) for resolution of naproxen (NAPROSYN®, a trademark of Syntex, Inc.) (over 1000 tons per year) as a substitute for cinchonidine. Recrystallization of diastereomeric salts usually need polar solvents such as alcohols, acetone with varying degrees of water (Ref. 4).

Some references of interest include:

U. C. Dyer, et al., Org. Proc. Res. Dev. 3 (#3), 161-165 (1999). This article discusses the application of automation and thermal analysis for resolving agent selection. However, it does not teach the present invention.

D. R. Aztec, et al., Adv. Synth. Catal. 345 (#4), 524-532 (2003). This article discusses automated enzyme screening methods for the preparation of enantiopure pharmaceutical intermediates. It is understood that U.S. Pat. Nos. 6,296,673 and 6,630,006, and others cited for high throughput transfer are modified in the present invention to add racemate to the individual tubes, thus increasing the efficacy of the identification of the condition and process.

B. D. Santarsieno, et al., in U.S. Pat. Nos. 6,296,673, 6,630,006, and related issued patents. These patents teach the use of high throughput automated screening of materials, primarily protein for optimal crystal growth for x-ray diffraction study.

Y-Chem International of Cupertino, Calif. has produced and sold simple non-automated kits for the selection of resolving agents, solvents and conditions. See http//:www.ychem.com.

Chirality in Industry, A. N. Collins, ed. Vol. I. The Commercial Manufacture and Application of Optically Active Compounds, John Wiley & Sons, Inc, New York, 1991. Chirality in Industry, G. N. Sheldrake, ed, vol. II, Developments of the Commercial Manufacture and Application of Optically Active Compounds, John Wiley & Sons, Inc., New York, 1997.

All U.S. Patents are incorporated herein by reference in their entirety.

The present invention is an improvement on the rapid selection of resolving agents, solvents and conditions.

### SUMMARY OF INVENTION

The present invention relates to one or more kits for improved identification of the optimal conditions for diasteroisomeric salt crystallization and the selection of the optimal solvents and resolving agents, which kit comprises:
A. An array of containers wherein the array is a standard high throughput tray and the containers are a multiplicity of substantially identical containers or well plates each optionally sealed with a sealant or stoppers, to avoid loss of chemicals,
B. wherein each substantially identical container has a unique combination of resolving agent in each column and at least one unique suitable solvent in each row; and
C. Instructional text to use the kit.

The present invention also relates to a method for the rapid high throughput determination of the solvents and conditions for the crystallization of diasteroisomeric salts to separate enantiomers, which method comprises:
A. Obtaining the kit of claim 1;
B. Adding to each container of the array of claim 1:
   i. a measured amount of racemic organic compound neat
C. Heating the combination of sub-step C to a solubilization temperature not in excess of 100° C. for less than 15 minutes;
D. Optionally, agitating the combination of substep C for between about 5 min. and 24 hr.;
E. Cooling the heated combination of sub-step C;
F. Observing the formation of diastereomeric crystals visually or by optical means in each container;
G. Separating the formed diastereoisometric salts;
H. Isolating and evaluating the desired isomer; and
I. Selecting the optimal combination of resolving agent and solvents and resolution conditions based on the experimental results of substeps A to H.

### BRIEF DESCRIPTION OF THE FIGURES

FIG. 1A is a schematic representation of a flow chart for the separation (resolution) of racemic bases. For neutral racemates such as aldehydes, alcohols, ketones and the like, the same steps are used after the pre-processing steps described herein below in the identified section are performed.

FIG. 1B is a schematic representation of a flow chart for the resolution (separation) of racemic acids. For amino acids, the same steps are used after the pre-processing steps described herein below in the identified section are performed.

FIG. 2A is a photographic view of the top of a kit showing the columns and rows of tubes in the tray with a cover sheet.

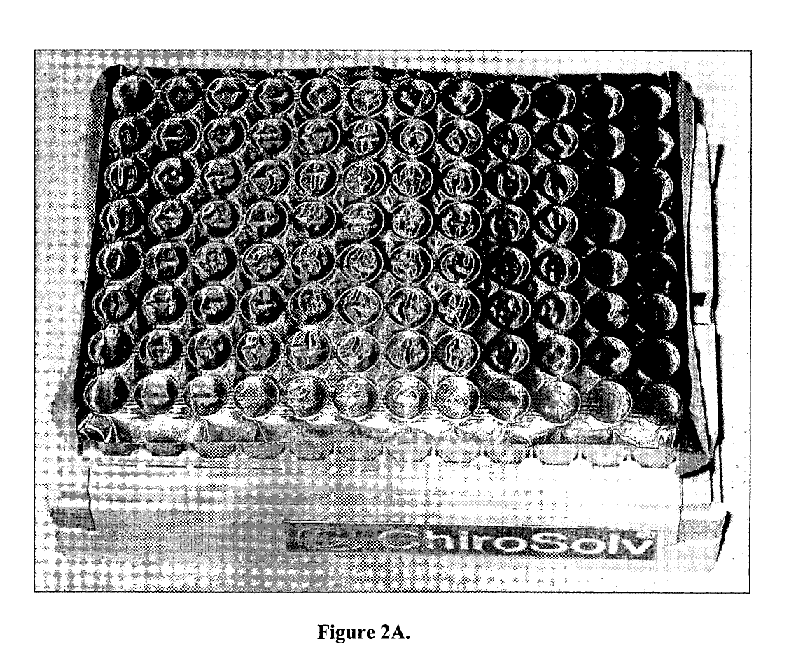

FIG. 2B is a photographic top view of the kit with tubes individually sealed with septum.

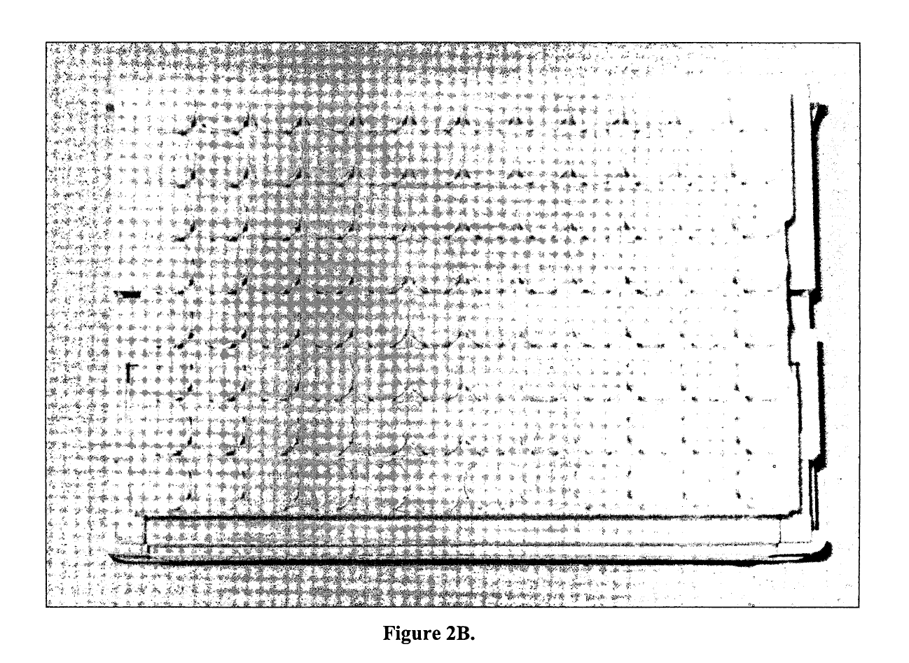

FIG. 3A is a photographic view of a side view of the tubes sealed with a sheet and tray of the kit.

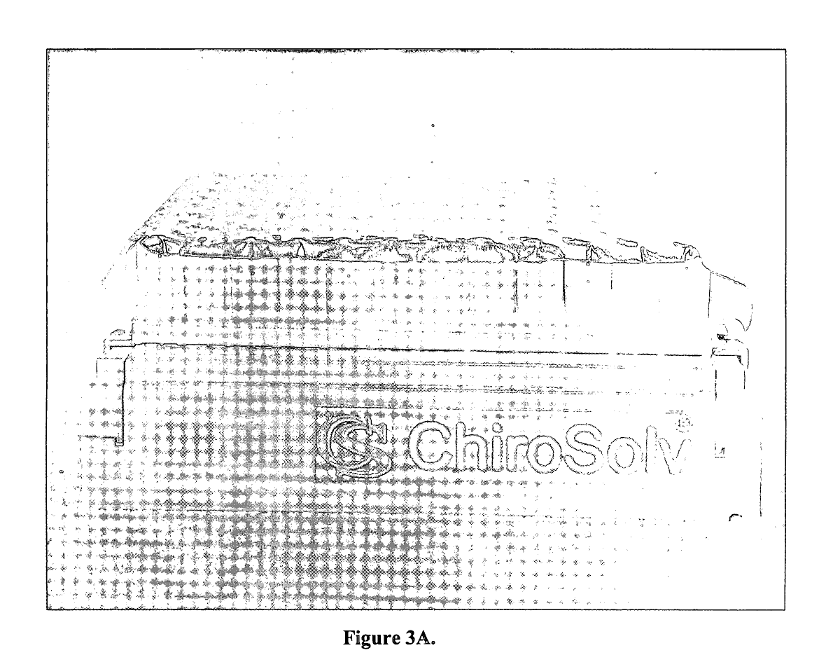

FIG. 3B is a photographic side view of the tubes individually sealed with septums in the tray.

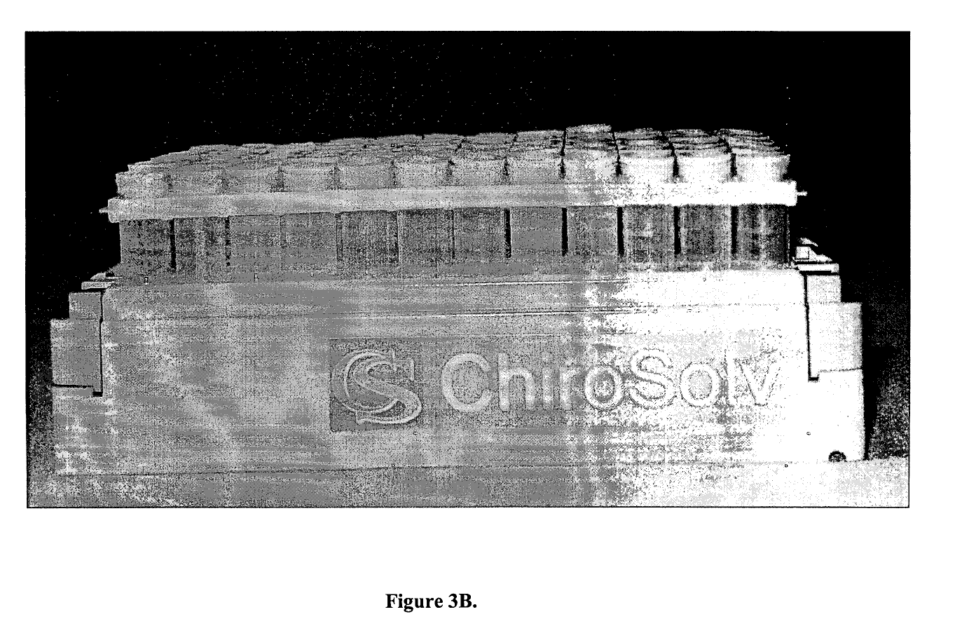

FIG. 4 is a photographic view of one tube having a bar code on the bottom and a second tube having an alphanumeric code.

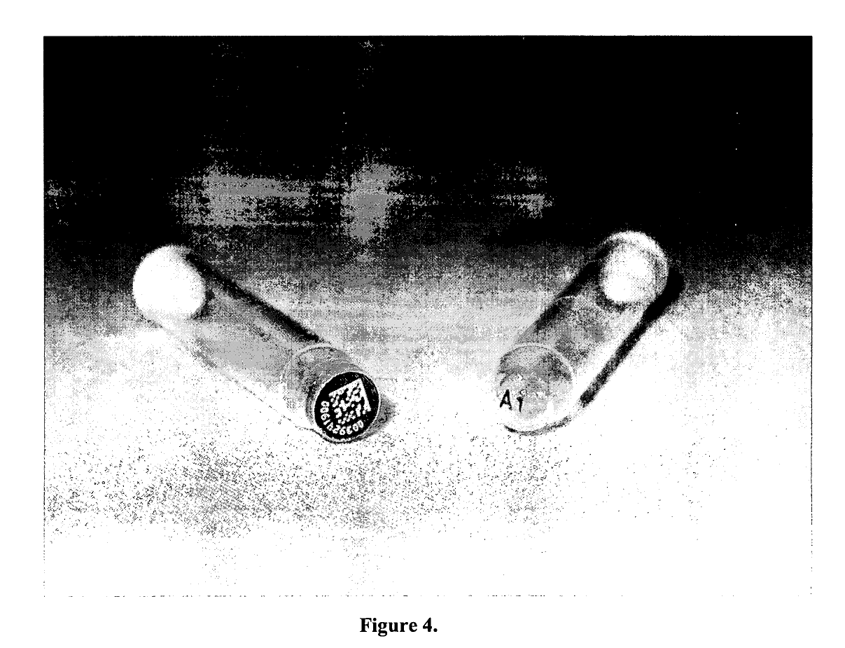

FIG. 5 is an isometric view of the tube in the tray with a bar code.

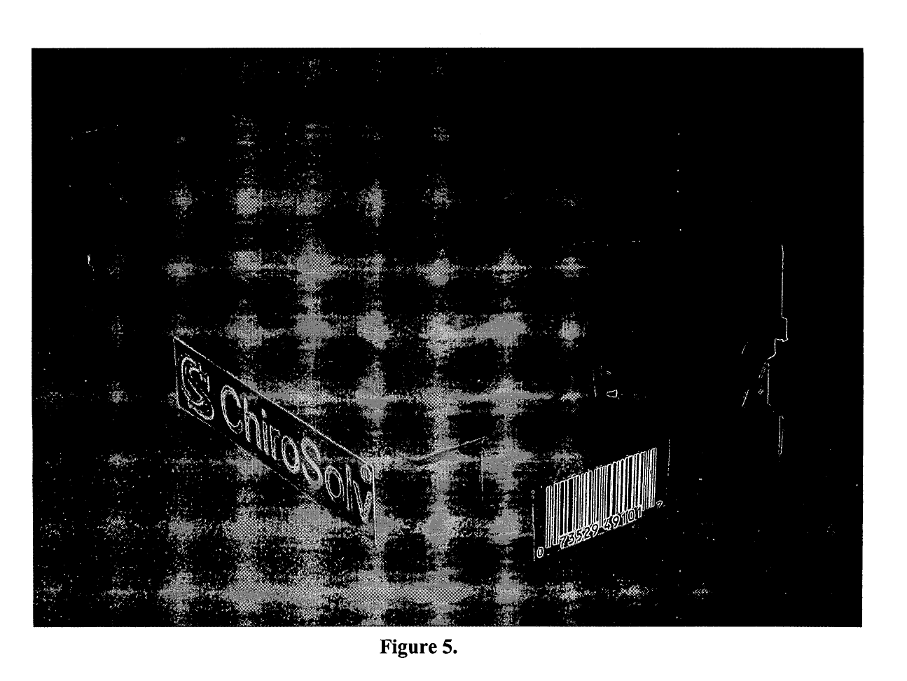

FIG. 6 is an isometric photographic view of 96 tubes with a representative bar code on their bottom (on the left) and 96 tubes having a representative alphanumeric code on their bottom (on the right).

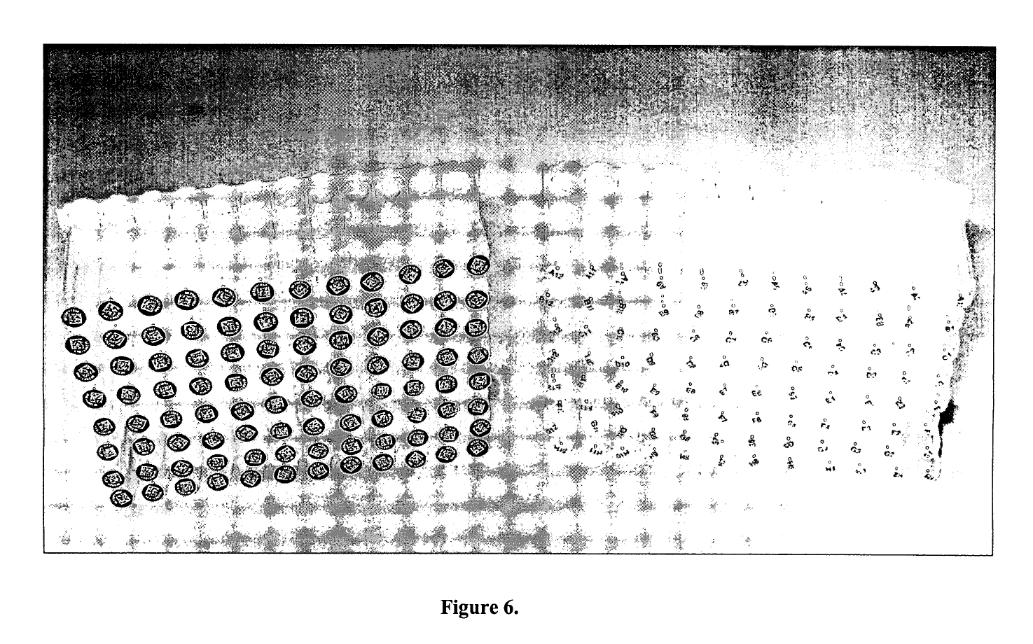

### DETAILED DESCRIPTION OF THE INVENTION AND PREFERRED EMBODIMENTS

### Definitions

As used herein:

“Acid resolving agent” or “acidic resolving agent” refers to commonly known acid resolving agents of the art. The resolving agent is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)—BOC-aminocyclopent-4-ene-1-(S)-carboxylic acid, 3-(S)—BOC-aminocyclopent-4-ene-1-(R)-carboxylic acid, N-2-BOC-D-arginine hydrochloride, N-2-BOC-D-aspartic acid, N-2-BOC-3-(4-bipheny)alanine, N-2-BOC—N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N—BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N—BOC-1,2,3,4-tetrahydroisoquinoline-(3R)-carboxylic acid, N—BOC-1,2,3,4-tetrahydroisoquinoline-(3S) -carboxylic acid, N-2-BOC-3-(4′-thiazolyl)alanine, N—BOC-D-threonine, N-2-BOC—N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N—CBZ-D-alanine, N—CBZ-D-arginine, N—CBZ-D-aspartic acid, N—CBZ-O-tert-butyl-D-serine, CBZ-1-cyclohexylalanine, N—CBZ-D-glutamic acid, N—CBZ-D-histidine, N—CBZ-D-leucine, N—CBZ-1-tert-leucine DCHA salt, N—CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50% solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine and combinations thereof.

“Base resolving agent” or “basic resolving agent” refers to commonly known base resolving agents of the art. These resolving agents include, but are not limited to N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, Bis {1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaninol, BOC-prolinol, N-butyl-2-amino-2-methyl-3-(3′,4′-dihydroxyphenyl)-propionate, CBZ-1-cyclohexylalaninol, N-2-CBZ-D-3-(1′naphthyl)alaninol, N-2-CBZ-D-3-(2′naphthyl)alaninol, N-1-phenylalaninol, 2-(2′-chlorobenzyl)benzyl-amine, 2-(3′-chlorobenzyl)benzyl-amine, 2-(4′-chlorobenzyl)benzylamine, (S)-cyclohexylalaninol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaninol, N-(3,4-dimethoxybenzyl)-1-phenylethylamine, 3,3-dimethyl-2-aminobutane, N,N-dimethyl-1-methylbenzylamine, N, N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl(R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl derivative, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaninol, (R)-1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

“Solvent” refers to those organic liquids (optionally in any combination with water) which solubalize the components. Solvents include, but are not limited to 90% acetone, methyl ethyl ketone (2-butanone), 1-butanol, 2-propanol, 90% 2-propanol, methanol, 80% methanol, ethanol, 96% ethanol, water, 1-propanol, 85% 1-propanol, acetonitrile, ethyl acetate, dichloromethane, chloroform, p-dioxane, methyl-t-butyl ether, toluene, tetrahydrofuran. The kit may also utilize one or more solvents selected from the group consisting of 1-butanol, 2-butanol, n-butyl acetate, carbon tetrachloride, chlorobenzene, chloroform, cyclohexane, cyclopentane, o-, m-, p-dichlorobenzene, dimethyl acetamide, dimethyl sulfoxide, dioxane, 2-ethoxyethanol, ethylene dichloride, glyme, heptane, hexadecane, hexane, iso-hexanes, 2-methoxyethanol, methyl t-butyl ether, methyl isoamyl ketone, methyl n-propyl ketone, dichloromethane, N-methylpyrrolidine, nonane, pentane, petroleum ether, propylene carbonate, pyridine, tetrahydrofuran, toluene, benzene, trichloroethylene, 1,1,2-trichlorotrifluoroethane, 2,2,4-trimethylpentane, o-xylene, actal, acetamide, acetophenone, acetylacetone, adiponitrile, allyl acetate, allyl alcohol, anisole, benzenethiol, benzonitrile, benzyl acetate, benzyl alcohol, benzyl benzoate, benzyl chloride, benzyl ethyl ether, bis(2-chloroethyl)ether, bis (2-ethylhexyl acetate), bromobenzene, 1-bromobutane, 2-bromobutane, 1-bromo-2-chloroethane, bromochloromethane, 1-bromodecane, 2-bromo-2-methylproprane, 1-bromonaphthalene, 1-bromopentane, 1-bromopropane, 2-bromopropane, 1,3-butanediol, 1,4-butanediol, 2,3-butanediol, butanenitrile, butanethiol, cis & trans 2-butene-1,4-diol, butyl acetate, sec-butyl acetate, tert-butyl benzene, butyl ethyl ether, butyl formate, butyl methyl ketone, butyl stearate, p-tert-butyltoluene, butyl vinyl ether, γ-butyrolactone, 1-chloro-3-methylbutane, 3-(chloromethyl)heptane, 1-chloronaphthalene, 1-chlorooctane, 1-chloropentane, o-, m-, p-chlorotoluene, cineole, o-, m-, p-cresol, cis,trans-crotonyl alcohol, cumene, cyclohexaol, cyclohexanone, cyclohexene, cyclohexylbenzene, cyclopentanone, p-cymene, cis,trans-decahydronaphthalene, decane, 1-decene, diacetone alcohol, dibenzyl ether, 1,2-dibromo-1,1-difluoroetane, 1,2-dibromoethane, dibromofluoromethane, dibromomethane, 1,2-dibromopropane, dibutyl ether, dibutyl maleate, dibutyl phthalate, dibutyl sebacate, dibutyl sulfide, 1,2-dichloropropane, 2,4-dichlorotoluene, 3,4-dichlorotoluene, diethyl carbonate, diethylene glycol, diethylene glycol dibutyl ether, diethylene glycol, diethylene glycol diethyl ether, diethylene glycol dimethyl ether, dimethylene glycol monoethyl ether, diethylene glycol monoethyl ether acetate, diethylene glycol monomethyl ether, diethyl ketone, diethyl malonate, diethyl oxalate, 2,3-diethylpentane, diethylpentane, diethyl sulfide, diiodomethane, diisobutyl ketone, dipentyl ether, diisopropyl ether, diisoprpyl ketone, dimethyl adipate, dimethyl aniline, 2,2-dimethylbutane, 2,3-dimethylbutane, 3,3-dimethyl-1-butanol, 2,3-dimethyl-2-butanol, 3,3-dimethyl-2-butanol, cis,trans-1,2-dimethylcyclohexane, dimethyl disulfide, N,N-dimethylformamide, dimethyl glutarate, 2,2-dimethylheptane, 2,2-dimethylhexane, 2,3-dimethylhexane, 2,4-dimethylhexane, 2,5-dimethylhexane, 3,3-dimethylhexane, 3,4-dimethylhexane, dimethyl maleate, 1,2-dimethylnaphthalene, 1,6-dimethylnaphthalene, 2,2-dimethylpentane, 2,3-dimethylpentane, 2,4-dimethylpentane, 3,3-dimethylpentane, dimethyl phthalate, 2,2-dimethyl-1-propanol, dimethyl succinate, 1,3-dioxolane, dipentene, dipentyl ether, diphenyl ether, dipropyl ether, dodecane, 1-dodecene, 1,2-epoxybutane, ethyl acetoacetate, ethyl acrylate, ethylbenzene, ethyl benzoate, ethyl butanoate, 2-ethyl-1-butanol, ethylbutyl ketone, ethyl trans-cinnamate, ethyl cyanoacetate, ethylcyclohexane, ethylene carbonate, ethylene glycol, ethylene glycol diacetate, ethylene glycol dibutyl ether, ethylene glycol diethyl ether, ethylene glycol dimethyl ether, ethylene glycol ethylether acetate, ethylene glycol monomethyl ether acetate, ethylene glycol monobutyl ether, ethylene glycol monoethylether, ethylene glycol monomethyl ether, 3-ethylhexane, 2-ethyl-1,3-hexanediol, 2-ethyl-1-hexanol, 2-ethylhexyl acetate, ethyl isovalerate, ethyl lactate, 3-ethyl-2-methylpentane, 3-ethyl-3-methylpentane, 3-ethylpentane, ethyl propanoate, fluorobenzene, o-, m-, p-fluoro-toluene, formamide, furfuryl alcohol, glycerol, heptane, 1-heptanol, 2-heptanol, 3-heptanol, 1-heptene, cis,trans2-heptene, hexafluorobenzene, hexamethylphosphoric trimide, hexane, hexanenitrile, 1,2,6-hexanetriol, 1-hexanol, 2-hexanol, 3-hexanol, 1-hexene, cis,trans-2-hexene, cis,trans-3-hexene, hexyl acetate, sec-hexyl acetate, hexylene glycol, hexyl methyl ketone, hydraacrylonitrile, iodobenzene, 1-iodobutane, 2-iodobutane, iodoethane, 1-iodo-2-methylpropane, 1-iodopropane, 2-iodopropane, isobutyl acetate, isobutylbenzene, isobutyl formate, isobutyl isobutanoate, isopentyl acetate, isopentyl isopentanoate, isophorone, isopropyl acetate, D & L-limonene, 2,4-lutidine, 2,6-lutidine, mesitylene, mesityl oxide, n-methylacetamide, methyl acetate, methyl acetoacetate, methyl benzoate, 2-methyl-1-butanol, 3-methyl-1-butanol, 2-methyl-2-butanol, 3-methyl-2-butanol, methyl cyanoactate, methylcyclohexane, 1-methylcyclohexanol, cis,trans-2-methylcyclohexanol, cis,trans-3-methylcyclohexanol, cis,trans-4-methylcyclohexanol, methylcyclopentane, N-methylformamide, 2-methylheptane, 3-methylheptane, 4-methylheptane, 2-methylhexane, 3-methylhexane, methyl isobutyl ketone, methyl isopentyl ketone, 1-methylnaphthalene, 2-methyloctane, 3-methyloctane, 4-methyloctane, methyl oleate, 2-methylpentane, 3-methylpentane, 2-methyl-1-pentanol, 3-methyl-1-pentanol, 2-methyl-2-pentanol, 3-methyl-2-pentanol, 4-methyl-2-pentanol, 2-methyl-3-pentanol, 3-methyl-3-pentanol, 4-methyl-4-penten-2-one, methyl pentyl ketone, N-methylpropanamide, 2-methylpropanenitrile, 2-methyl-1-propanol, 2-methyl-2-propanol, methyl propyl ketone, N-methyl-2-pyrrolidone, methyl salicylate, 2-methyl tetrahydrofuran, 2-methylthiophene, 3-methylthiophene, 4-methylvaleronitrile, β-myracene, nitroethane, nitromethane, 1-nitropropane, 2-nitropropane, nonane, 1-nonene, octane, octanenitrile, 1-octanol, 2-octanol, 1-octene, cis,trans-2-octene, pentachloroethane, 1,5-pentanediol, pentanenitrile, 1-pentanol, 2-pentanol, 3-pentanol, pentyl acetate, β-phellandrene, phenetole, 2-picoline, 3-picoline, 4-picoline, α-pinene, β-pinene, 1,2-propanediol, 1,3-propanediol, propanenitrile, propargyl acetate, propargyl alcohol, propyl acetate, propylbenzene, propyl benzoate, propylene carbonate, propyl formate, pseudocumene, styrene, α-terpinene, terpinolene, 1,1,2,2-tetrabromoethane, 1,1,1,2-tetrachloroethane, 1,1,2,2-tetrachloroethane, tetrachloroethylene, tetrachloromethane, tetraethylene glycol, tetraethylsilane, tetrahydrofuran, tetrahydrofurfuryl alcohol, tetrahydronaphthalene, tetrahydropyran, tetrahydrothiophene, 2,2,3,3-tetramethylpentane, 2,2,3,4-tetramethylpentane, 2,2,4,4-tetramethylpentane, 2,3,3,4-tetramethylpentane, tetramethylurea, thiodiethanol, thiophene, toluene, o-, m-, p-toluidine, α-tolylnitrile, triacetin, tribromomethane, tributyl borate, tributyl phosphate, 1,1,1-trichloroethane, 1,1,2-trichloroethane, trichloroethylene, trichloroethylsilane, trichlorofluoromethane, (trichloromethyl)benzene, trichloromethylsilane, 1,2,3-trichloropropane, 1,1,2-trichlorotrifluoroethane, tri-o-cresyl phosphate, tridecane, 1-tridecene, triethylene glycol, triethyl phosphate, 2,2,2-trifluoroethanol, (trifluoromethyl)benzene, 1,2,3-trimethylbenzene, 2,2,3-trimethylbutane, 2,2,5-trimethylhexane, 2,3,5-trimethylhexane, 2,2,3-trimethylpentane, 2,2,4-trimethylpentane, 2,3,3-trimethylpentane, 2,3,4-trimethylpentane, trimethyl phosphate, 1-undecene, veratrole, vinyl acetate o-, m-, p-xylene and combinations thereof.

The present invention permits one of skill in the art to quickly screen resolving agents and solvents to find the most optimum combination and to optimize reaction conditions in order to separate a racemic mixture (acids, bases, alcohols, amino acids, aldehydes/ketones) into its constituent enantiomers. It does this by offering six types of kits, each with, for example, 12 rows of 8 vials (a total of 96 vials). Each vial contains a pre-measured quantity of a unique combination of resolving agent and solvent. As a result, scientists can potentially screen up to 576 combinations of the resolving agents and solvents, if all six types of kits are used at the same time.

Resolving agents are chosen with manufacturing use in mind. They are relatively inexpensive and readily recoverable in high yield after completion of the resolution. In industrial practice, the quantity of resolving agent is often less than the stoichiometric amount, which allows for better separation of the desired enantiomer at a lower cost.

By providing pre-measured quantities of resolving agents and solvents, the present invention offers:
a) the ability to do research in parallel, reducing the research time by up to 90%, i.e., experiments can be finished in days rather than months;
b) technology know-how; resolving agents and the solvents and their proportions are selected with full understanding of the solubility diagram and through years of experience in developing chirally pure compounds by the chief technologist;
c) consistent research environment and accurate results;
d) resolving agents are chosen with manufacturing in mind; which are relatively inexpensive and readily recoverable in high yield after completion of the resolution;
e) optimized use of the skilled staff time by avoiding mundane mechanical work of measuring; and
f) elimination of human errors. The kits are designed to be used with auto-station; each vial and kit has identification barcode for easier tracking

Each disposable kit is equipped with plastic vials (e.g. 96) that bear unique alphanumeric and/or barcode markings and are held in a rack designed for robotic manipulation. Vials are usually about 0.75 to 4 ml, preferably about 1.4 ml in size, thus requiring very small amount of the unknown sample. Both vials as well as the rack are heat and chemical resistant and withstand temperatures of −20° to +120° C. This unique design allows the scientists to perform the entire experiment without having to take the vials out of the rack.

The advantages of the present invention include:
a) six types of kits providing 576 combinations (or more kits possible) of solvents and resolving agents;
b) ready-to-use disposable kits (all you need is your unknown compound);
c) very little amount of unknown needed (less than 3 mmol per kit);
d) vials and the racks both have the same material specificity, making them heat and chemical resistant, and able to withstand temperatures of −20′ to +120° C. (the entire experiment can be done within the rack);
e) easy to follow step-by-step instructions and results charts;
f) conveniently designed for easy robotic manipulations to eliminate human errors;
g) kits and/or vials have unique identification (e.g., barcode or alphanumeric code) for cross-referencing; and
h) long shelf life for the kits.

These kits are based on a straightforward acid-base neutralization technique, followed by re-crystallization in suitable solvent. The goal is to determine the most optimum combination of resolving agent and solvent that allows quick crystallization of the chirally pure compound and to stipulate the conditions permitting maximal recovery of the pure enantiomer. The kits are primarily of two types:
Acid kits (e.g., A1, A2, A3, etc.): Include a group of chirally pure acids. They are used to resolve racemic bases. Each kit includes 8 types of acids and twelve types of solvents. Most of the acids used in these kits are easily available in bulk quantities and are commonly used in manufacturing processes.
Base kits (e.g., B1, B2, B3, etc.): Include a group of chirally pure bases (or amines). They are used to resolve racemic acids. Each kit includes 8 types of amines and twelve types of solvents. Most of the bases used in these kits are easily available in bulk quantities and are commonly used in manufacturing processes.

Each experiment needs about 0.001 mmol to 0.1 millimol, preferably about 0.03 mmol of unknown racemate in each of the 96 vials. The mixture is then heated close to the boiling point of the solvent and then allowed to cool at ambient temperature. It is then further cooled to 4° C. and finally to 0° C. All vials with crystals are indicators of success; while the rest of the vials need to be examined for quick excess solvent test (the lack of crystals may be due to excess quantity of solvent). Typically, only one or two vials will show maximum optical purity. One would be dextro (+) and other laevo (−) rotatory to sodium light. From this matrix, usually only one vial with desired enantiomer will have to be investigated further in addition to its mother liquor for scale-up condition optimization.

The screening experiments involve following steps:
1. Choose the correct type of kit (A1, A2, A3, etc. or B1, B2, B3, etc.) depending on whether the unknown racemate is base or acid respectively.
2. If the racemate is a type of alcohol, amino acid, aldehyde or ketone, then the pre-processing as is described below is needed.
3. Add 0.01 to 0.05 mmol of the racemate to each of 96 vials. Depending on the availability of the dispensing autostation and the racemate type (liquid or powder), one may need to remove the vial caps. Note that the caps are pre-slitted to accommodate direct injection of racemate by a conventional autostation of the art.
4. Heat the rack and its vials to about 80° C. (the optimum temperature for most of these experiments) or until the mixture becomes homogeneous (up to 100° C.).
5. Allow the kit to cool normally to ambient temperature. Next, if required, further cool the kit to about 4° C. and finally to about 0° C. and observe any crystallization. Vials with crystals formed are considered to be positive tests and need further investigation.
6. Using crystal initiation techniques, attempt to obtain more vials with crystals scratching, seed crystal of enantiomer, etc. Vials with no crystals even after this further action are considered negative tests.
7. Separate the vials with crystals (positive tests) and note their barcode, alphanumeric code, or combination thereof for identification.
8. Analyze each of the crystals separately after liberating enantiomers from the respective diastereomeric salts for specific rotation measurement to sodium or mercury light.
   Pre-Processing for Alcohols

An alcohol is neutral in functionality and it is usually resolved by conventional conversion to the mono-ester of succinic or phthalic acid. This hydrogen succinate or phthalate is then converted into diastereomeric salt by contact with optically active bases as described herein.

The pre-steps include:
1. Treat the racemic alcohol with 1×1 molar ratio of phthalic anhydride and greater than 1×1 molar ratio of pyridine. It is permitted to use 1×1 molar ratio of succinic anhydride instead of the pyridine.
2. Heat the mixture to between about 80 to 100° C. for 2 hrs.
3. Cool the mixture to ambient temperature and then quench with ice water containing sufficient sulfuric acid to make the whole mixture acidic, i.e., a pH less than 7. This mixture will be the hydrogen phthalate, either in the form of an oil or as a crystalline solid. If the mixture is oil, treat it with acetone and/or use conventional crystal initiation techniques as necessary to crystallize it.
4. Filter, wash and then dry the mixture. The product of the hydrogen phthalate with free having a carboxyl function.
5. Use the kits B1, B2, B3, etc. as described above.

Pre-processing for Amino Acids (Amphoteric Racemate)

Amino acids exist in Zwitter ion (dual charged) structure. A synthetic amino acid is primarily resolved using one of the following two types of methods:
1. By protecting the carboxyl group (and freeing the amino group), usually using esterification, or
2. By protecting the amino group (and freeing the carboxyl group), usually using formylation.
   Protection of Carboxylic Group Using Esterification:

The carboxyl end of the molecule is protected by standard esterification using mild base and an alcohol followed by diastereomeric salt formation of the free amine function and needs screening kits made up of chiral acids. Many racemic alpha-amino acids have been successfully resolved by preparing the corresponding isobutyl ester or benzyl ester.

Steps:
1. Add a sufficient amount of dilute HCl to the racemate to dissolve it and adjust the pH to 3.
2. Cool the mixture to between about 0 to 2° C.
3. Esterify by adding (1:1.2 ratio) of isobutyl ester or benzyl ester.
4. Heat the mixture to 100° C. and then cool it to 0 to 5° C.
5. Decrease the acidity to pH 7 by adding NaOH.
6. Use the kits A1, A2, A3, etc. as described above to obtain the diastereomeric salt.
7. After having identified the ideal candidate vial containing crystals, then remove the ester group introduced in step 3 under mild acid hydrolysis conditions and verify that no racemization has occurred.
   Protection of Amino Group Using Formylation:

The carboxylic group is then screened with the amines kits (B1, B2, B3, etc.). After having identified the ideal candidate vial, one then removes the formyl group under mild hydrolysis conditions and verifies that no racemization has occurred.

Steps:
1. Add a sufficient amount of 1N NaOH solution to the racemate to dissolve it and bring the pH to 10.
2. Cool the mixture to 0 to 2° C.
3. Formylate by adding (1:1.2 ratio) of triethyl orthoformate.
4. Heat the mixture to 100° C. and then cool it to 0 to 5° C.
5. Increase the acidity to pH 4 by adding hydrochloric or sulfuric acid.
6. Use the kits B1, B2, B3, etc. as described above.
7. After having identified the ideal candidate vial, one should then remove the formyl group introduced in step 3 under mild acid hydrolysis conditions and verify that no racemization has occurred
   Preprocessing for Aldehydes and Ketones

In order to be resolved by salt formation, aldehydes and ketones must be transformed into either acidic or basic derivatives.

Acidic Derivatives:

Reagents such as 4-sulfonylphenylhydrazine, 4-(4-carboxyphenyl)semicarbazide, 4-hydrazinobenzoic acids (para/meta), oxalic acid monohydrazide is used. These salts are then be resolved by chiral bases.

Steps:
1. Treat the racemic aldehyde or ketone in minimum amount of methanol
2. Cool the mixture to 0° to 5° C.
3. Add one of the above-cited reagents. (The result is a crystalline protected amino acid.)
4. Isolate the protected amino acid using filtration or centrifugation.
5. Use the kits B1, B2, B3, etc. as described above
   Basic Derivatives:

A carbonyl can be converted into enamine using tertiary amines, which enamine is then resolved by chiral acids. Alternatively, carbonyl is treated with bisulphite salts of chiral amines, and resulting diastereomers are separated by crystallization.

Steps:
1. Treat the racemic aldehyde or ketone in minimum amount of methanol.
2. Cool the mixture to 0 to 5° C.
3. Add tertiary amine like pyrrolidine or piperidine. Alternatively one adds sodium bisulphite. (The result is a crystalline protected amino acid.)
4. Isolate the protected amino acid using filtration or centrifugation.
5. Use the kits B1, B2, B3, etc. as described above.
   Diastereomeric Crystallization Technique

Chirally-pure isomers are obtained through a variety of techniques. The most commonly used one is the classic resolution by diastereomeric crystallization. Because of its easy adoption in a manufacturing setting, most companies try this approach first; and then use other approaches only if this one fails. Currently, over 65% of all chiral products are developed using this technique.

The primary reasons for preferring diastereomeric crystallization in manufacturing are economic, that is:
a) It is easier and therefore cheaper to build up the racemate needed for resolution methods than it is to create pure isomers using the synthetic technologies.
b) Among the resolution techniques available, resolution by diastereomeric crystallization is less time and temperature sensitive and less complex.
c) The equipment for doing diastereomeric crystallization is more likely to already exist in manufacturing installations.
d) Racemization in connection with diastereomeric crystallization ultimately produces a high yield of the enantiomer much more cheaply than the other resolution or synthetic procedures. (Racemization is the process of repeatedly reprocessing the “waste” portion of the resulting products; each subsequent pass yielding additional good product)
e) Resolution by diastereomeric crystallization is also generally superior to enzymatic resolution in that it usually yields a product of higher enantiomeric purity and both isomers are separated. In enzymatic resolution, one isomer is usually destroyed. Also, enzyme resolution generally does not yield a highly pure (ee) isomer and thus one needs to utilize diastereomeric crystallization as the last step.
   Resolving Agents

A classical resolving agent is a chiral acid or base (optically active isomer, enantiomer), which has a propensity to form a crystalline diastereomer when combined with a racemic base or acid. Some requirements of an ideal resolving agent include:
1. Proximity of stereogenic centers,
2. Rigid structure,
3. Must have strong acid or base characteristics,
4. Must have chemical and optical stability,
5. Both enantiomers must be available and recyclable, and
6. Must be availability in bulk quantities at relatively low price.

Amines and cinchonal alkaloids found typically in natural products meet these requirements and are used most often.

Resolution of Different Materials

For resolving carboxylic acids one usually forms salts with optically active amines. On the other hand, for resolving amine: one uses enantiomeric pure acids such as tartaric acid, malic acid and mandelic acid.

To resolve neutral compounds, one prepares covalent diastereomeric derivatives. e.g. with alcohols, one forms monophthalate, succinate or ester; while with ketones, one forms the corresponding hydrazones.

Resolution of Amino Acids (Amphoteric Racemates)

Amphoteric racemates have both acidic and basic characteristics, e.g., in aspartic acid, there are two carboxylate groups for one amine group. The compound is resolved as a simple acid or base. For compounds having one carboxyl and amino group each, one of the functional group must be functionalized.

Resolution of Neutral Compounds

If resolution of a neutral compound by salt formation is intended, the compound must first be transformed to a derivative containing an acidic or basic group. Resolution by derivatization is typical for alcohols, aldehydes and ketones. Alcohols are almost exclusively transformed to their monophthaletes or succinates. Usually phthalates (phthalic or 3-nitrophthalic anhydride) or succinic anhydride for succinates are used.

The inherent low yields of resolution are increased to nearly 100% using various techniques. The best resolutions are those in which the undesirable enantiomer is later racemized and recycled to produce overall yields close to 100%.

The following examples are provided for description and explanation only. They are not to be construed to be limiting in any way.

### EXAMPLES

General

The solvents are available from commercial sources, usually as reagent-grade and used without further modification.

The acid and base resolving agents are available from commercial sources and are used without further purification.

The reagents to transform a “neutral” precursor compound to a useful derivative are available from commercial sources and are used without purification.

The kits (tray and tube combinations) are available from the inventor as CHIROSOLV of Cupertino, Calif. (See http//:www.chirosolve.com)

Commercial chemical suppliers include, but are not limited to, Aldrich Chemical, Milwaukee, Wis., MP Biomedicals, Irvine, Calif., etc.

Additional sources are located in Chemical Sources USA, published annually by Chemical Sources International, Inc. of Clemson, S.C., 29633.

Solvent and chemical commercial sources are also found in Chemical Sources, USA at www.chemsources.com.

The tubes or containers, stoppers, film, etc. are commercially available from chemical supply houses such as E & C Scientific, Inc.; Matrix Technologies, Inc., Hudson, N.H.; Abgene, Inc., Rochester, N.Y.; TomTec, Inc., Hamden, Conn.; and Micronic Mass., McMurray, Pa. The object is a commercial film having adhesive or quasi-adhesive properties. Usually it is a polymer, aluminum, and/or combinations thereof. The sheet is useful to retain solvents and resolving agents prior to use. The commercial septums perform the same function for the individual containers.

The tubes or containers with the bar code or alphanumeric code labels are made from commercially available makers for example, Matrix, Inc., Abgene, Inc.

### Example 1

### Resolution of Racemic Acid Using ChiroSolv® Kit

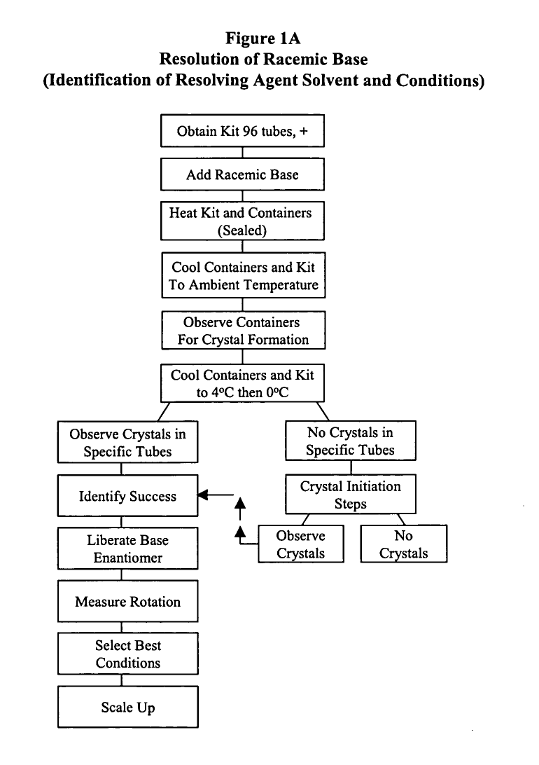

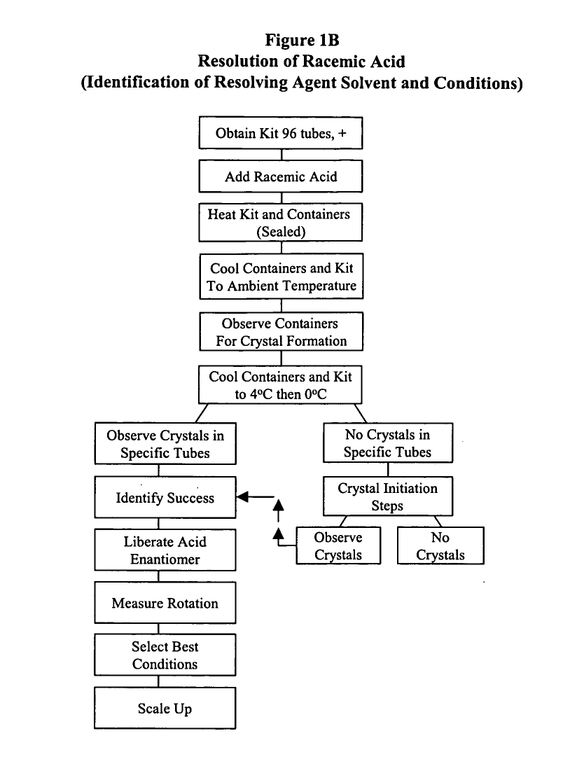

### This procedure corresponds in general to FIG. 1B

1. Use kits B1, B2, B3 or combination thereof (preferably all of them for best results).
2. Remove the lid of the kit(s).
3. Determine if the unknown racemate acid is solid/powder.
   a) If yes, remove the seal of the kit and dispense about 0.01 to 0.03 mmol of unknown racemate into each container of the kit. Cover the containers with additional seal/rubber septa provided. Go to step 4.
   b) If no, dispense about 0.01 to 0.03 mmol of the liquid racemate into each container.
4. Heat the kit and containers and the mixture to 80° C., or until the mixture becomes homogeneous (up to 100° C.).
5. Optionally agitate the kit to encourage homogenization.
6. Cool the kit with containers and mixtures to ambient temperature.
7. Determine if any crystals formed.
   a) If yes, select the containers with crystals, close them with additional rubber septum provided and set them aside for further analysis. Go to step 11.
   b) If no, proceed to step 8.
8. Cool the kit with containers and mixtures further to 4° C. and then to 0° C.
9. Optionally use crystal initiation technique to encourage crystal formation.
10. Determine if any crystals formed.
   a) If yes, select the containers with crystals, close them with additional rubber septum provided and set them aside for further analysis. Go to step 11.
   b) If no, discard the containers without the crystals and exit.
11. Note the identification marking (bar code or alphanumeric code) of the kit as well as the individual containers that are to be analyzed further.
12. Analyze the crystals of each container selected separately after liberating enantiomers from the respective diastereomeric salts for specific rotation measurement using sodium or mercury light.
13. Select one or two containers out of all containers in step 12 with crystals that have maximum optical purity. These are the optimal resolving agent and solvent combinations for the given racemate.
14. Evaluate the selected crystals in step 13 with its mother liquor for scale-up condition optimization.

### Example 2

### Resolution of Racemic Bases Using ChiroSolv® Kit

### This Procedure Corresponds in General to FIG. 1A

1. Use kits A1, A2, A3 or combination thereof (preferably all of them for best results).
2. Remove the lid of the kit(s).
3. Determine if the unknown racemate base is solid/powder.
   a) If yes, remove the seal of the kit and dispense about 0.01 to 0.03 mmol of unknown racemate into each container of the kit. Cover the containers with additional seal/rubber septa provided. Go to step 4.
   b) If no, dispense about 0.01 to 0.03 mmol of the liquid racemate into each container.
4. Heat the kit and containers and the mixture to 80° C., or until the mixture becomes homogeneous (up to 100° C.).
5. Optionally agitate the kit to encourage homogenization.
6. Cool the kit with containers and mixtures to ambient temperature.
7. Determine if any crystals formed.
   a) If yes, select the containers with crystals, close them with additional rubber septum provided and set them aside for further analysis. Go to step 11.
   b) If no, proceed to step 8.
8. Cool the kit with containers and mixtures further to 4° C. and then to 0° C.
9. Optionally use crystal initiation technique to encourage crystal formation.
10. Determine if any crystal formed.
   a) If yes, select the containers with crystals, close them with additional rubber septum provided and set them aside for further analysis. Go to step 11.
   b) If no, discard the containers without the crystals and exit.
11. Note the identification marking (bar code or alphanumeric code) of the kit as well as the individual containers that are to be analyzed further.
12. Analyze the crystals of each container selected separately after liberating enantiomers from the respective diastereomeric salts for specific rotation measurement using sodium or mercury light.
13. Select one or two containers out of all containers in step 12 with crystals that have the maximum optical purity. These are the optimal resolving agent and solvent combinations for the given racemate.
14. Evaluate the selected crystals in step 13 with its mother liquor for scale-up optimization.

Our statistical screening resolving agents and optimization of resolution conditions are systematically studied by the present description in the chiral kit experiment.

Typical results of screening experiments are given below:

TABLE 4
Resolution of (±) Phenylpropionic acid (Hydratropic acid)

| Amines | Ethanol | 96 EtOH | MeOH | 80 MeOH | Eacetatate | 70 IPA | 99 IPA | 1-butanol |
|---|---|---|---|---|---|---|---|---|
| Phenethyl | Oil | Oil | Oil | Oil | α = −5.1 | Oil | Oil | α = 0 |
| Megluca- | α¹ = +4.0 | α = +3.0 | Oil | Oil | Oil | Oil | Oil | Oil |
| Strychnin | α = +7.0 | α = +7.5 | α = +5.2 | α = +5.5 | Oil | Oil | Oil | Oil |
| Quinidin | α = −3.0 | α = −5.2 | Oil | Oil | α = +2.0 | α = 0 | Oil | Oil |
| Quinin | α = +5.0 | α = +4.5 | Oil | α = +2.0 | Oil | Oil | Oil | Oil |
| Brucine | Oil | Oil | Oil | Oil | α = −1.2 | Oil | Oil | Oil |

αethanol(C = 0.1)
¹= Has to be cooled to 4° C.
Note:
All cells with oil indicates negative test that you should discard (after usual crystallization efforts)
Above table shows that the strychnine in 96% Ethanol is ideal system for (+) isomer, while quinidine in 96% Ethanol would be good system for (−) isomer.

TABLE 5
Resolution of (±) Phenylpropionic acid (Hydratropic acid)

| Acids | Ethanol | 96 EtOH | MeOH | 80 MeOH | Eacetatate | 70 IPA | 99 IPA | 1-butanol |
|---|---|---|---|---|---|---|---|---|
| Tartaric | α = 0 | α = −0.5 | α¹ = +5.0 | Oil | NA² | Oil | α¹ = 0 | Oil |
| Pyrogluta | Oil | Oil | Oil | Oil | NA² | α = +15.5 | Oil | Oil |
| Malic | α = +6.0 | α = +10.0 | α = −1.0 | Oil | NA² | α = +6.5 | Oil | α¹ = −5.0 |
| Mandelic | Oil | Oil | Oil | Oil | Oil | Oil | Oil | Oil |
| Dtolytart | NA² | NA² | α = +14.5 | NA² | NA² | NA² | NA² | Oil |
| Camphor | Oil | Oil | Oil | Oil | Oil | Oil | Oil | Oil |

¹= Little solvent was evaporated to yield crystals
NA² = Did not go in solution even at 80° C.
α = Neat

As evident from above data (+) isomer of amine, pyroglutamic acid in 70% IPA is ideal system, for (−)isomer, malic acid in 1-butanol is the system of choice. The literature shows malic acid in ethanol was used to resolve the racemic amine.

There are a few new developments in the field: one is by a Roche group in the United Kingdom, which has used differential scanning calorimetry as a means of to identify diastereomeric salts with a clear eutetic composition that is needed for effective resolution (Ref. 5). They also utilized robots to synthesize diastereomeric salts and facilitated data analysis by developing resolution package. In process called “Dutch Resolution” a family of resolving agents is being used instead of single agent, for example tartaric acid family composed of dibenzoyltartaric acid, ditolyltartaric acid and tartaric acid. According to the authors when such a mixture is added to a solution of a racemic substrate, a crystalline salt usually precipitates immediately. In most cases the substrate contained in the precipitated salt is resolved to about 90-98% ee (Ref. 6).

The foregoing examples and description of preferred embodiments of the present invention are provided for the purposes of illustration and description. The examples and preferred embodiments, however, are not intended to be exhaustive or to limit the invention to the precise forms disclosed. Obviously, many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

I claim:

1. A kit for improved identification of the optimal conditions for diasteroisomeric salt crystallization and the selection of the optimal solvents and resolving agents, which kit comprises:
A. an array of containers wherein the array is a standard high throughput tray and the containers are a multiplicity of substantially identical containers or well plates each optionally sealed with a sealant or stoppers to avoid loss of chemical solvent,
B. wherein each substantially identical container has a unique combination of resolving agent in each column and at least one suitable solvent in each row; and
C. an instructional text to use said kit.

2. The kit of claim 1 wherein the array or tray has at least 24 containers.

3. The kit of claim 1 wherein the array or tray has at least 48 containers.

4. The kit of claim 1 wherein the array or tray is selected from arrays of the group of at least 96 containers, or 192 containers, or 384 containers or 512 containers.

5. The kit wherein the array of containers is optionally sealed to exclude air or liquid with at least one sealant.

6. The kit of claim 1 wherein the resolving agent, acid or base, is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)—BOC-aminocyclopent-4-ene-1-(S)-carboxylic acid, 3-(S)—BOC-aminocyclopent-4-ene-1-(R)-carboxylic acid, N-2-BOC-D-arginine hydrochloride, N-2-BOC-D-aspartic acid, N-2-BOC-3-(4-bipheny)alanine, N-2-BOC—N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N—BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N—BOC-1,2,3,4-tetrahydroisoquinoline-(3R)-carboxylic acid, N—BOC-1,2,3,4-tetrahydroisoquinoline-(3S)-carboxylic acid, N-2-BOC-3-(4′-thiazolyl)alanine, N—BOC-D-threonine, N-2-BOC—N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N—CBZ-D-alanine, N—CBZ-D-arginine, N—CBZ-D-aspartic acid, N—CBZ-O-tert-butyl-D-serine, CBZ-1-cyclohexylalanine, N—CBZ-D-glutamic acid, N—CBZ-D-histidine, N—CBZ-D-leucine, N—CBZ-1-tert-leucine DCHA salt, N—CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50% solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine, N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, bis {1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaninol, BOC-prolinol, N-butyl-2-amino-2-methyl-3-(3′,4′-dihydroxyphenyl)-propionate, CBZ-1-cyclohexylalaninol, N-2-CBZ-D-3-(1′naphthyl)alaninol, N-2-CBZ-D-3-(2′naphthyl)alaninol, N-1-phenylalaninol, 2-(2′-chlorobenzyl)benzyl-amine, 2-(3′-chlorobenzyl)benzyl-amine, 2-(4′-chlorobenzyl)benzylamine, (S)-cyclohexylalaninol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaninol, N-(3,4-dimethoxybenzyl)-1-phenylethylamine, 3,3-dimethyl-2-aminobutane, N,N-dimethyl-1-methylbenzylamine, N,N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl(R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzyl amine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaninol, (R)1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

7. The kit of claim 1 wherein the solvent is selected from the group consisting of water acetone, 2-butonone, 2-propanol, methanol, ethanol, 1-propanol, acetonitrile, ethyl acetate, 1-butanol, 2-butanol, n-butyl acetate, carbon tetrachloride, chlorobenzene, chloroform, cyclohexane, cyclopentane, o-, m-, p-dichlorobenzene, dimethyl acetamide, dimethyl sulfoxide, dioxane, 2-ethoxyethanol, ethylene dichloride, glyme, heptane, hexadecane, hexane, iso-hexanes, 2-methoxyethanol, methyl t-butyl ether, methyl isoamyl ketone, methyl n-propyl ketone, dichloromethane, N-methylpyrrolidine, nonane, pentane, petroleum ether, propylene carbonate, pyridine, tetrahydrofuran, toluene, benzene, trichloroethylene, 1,1,2-trichlorotrifluoroethane, 2,2,4-trimethylpentane, o-xylene, actal, acetamide, acetophenone, acetylacetone, adiponitrile, allyl acetate, allyl alcohol, anisole, benzenethiol, benzonitrile, benzyl acetate, benzyl alcohol, benzyl benzoate, benzyl chloride, benzyl ethyl ether, bis(2-chloroethyl)ether, bis(2-ethylhexyl acetate), bromobenzene, 1-bromobutane, 2-bromobutane, 1-bromo-2-chloroethane, bromochloromethane, 1-bromodecane, 2-bromo-2-methylproprane, 1-bromonaphthalene, 1-bromopentane, 1-bromopropane, 2-bromopropane, 1,3-butanediol, 1,4-butanediol, 2,3-butanediol, butanenitrile, butanethiol, cis & trans 2-butene-1,4-diol, butyl acetate, sec-butyl acetate, tert-butyl benzene, butyl ethyl ether, butyl formate, butyl methyl ketone, butyl stearate, p-tert-butyltoluene, butyl vinyl ether, γ-butyrolactone, 1-chloro-3-methylbutane, 3-(chloromethyl)heptane, 1-chloronaphthalene, 1-chlorooctane, 1-chloropentane, o-, m-, p-chlorotoluene, cineole, o-, m-, p-cresol, cis,trans-crotonyl alcohol, cumene, cyclohexaol, cyclohexanone, cyclohexene, cyclohexylbenzene, cyclopentanone, p-cymene, cis,trans-decahydronaphthalene, decane, 1-decene, diacetone alcohol, dibenzyl ether, 1,2-dibromo-1,1-difluoroetane, 1,2-dibromoethane, dibromofluoromethane, dibromomethane, 1,2-dibromopropane, dibutyl ether, dibutyl maleate, dibutyl phthalate, dibutyl sebacate, dibutyl sulfide, 1,2-dichloropropane, 2,4-dichlorotoluene, 3,4-dichlorotoluene, diethyl carbonate, diethylene glycol, diethylene glycol dibutyl ether, diethylene glycol, diethylene glycol diethyl ether, diethylene glycol dimethyl ether, dimethylene glycol monoethyl ether, diethylene glycol monoethyl ether acetate, diethylene glycol monomethyl ether, diethyl ketone, diethyl malonate, diethyl oxalate, 2,3-diethylpentane, diethylpentane, diethyl sulfide, diiodomethane, diisobutyl ketone, dipentyl ether, diisopropyl ether, diisoprpyl ketone, dimethyl adipate, dimethyl aniline, 2,2-dimethylbutane, 2,3-dimethylbutane, 3,3-dimethyl-1-butanol, 2,3-dimethyl-2-butanol, 3,3-dimethyl-2-butanol, cis,trans-1,2-dimethylcyclohexane, dimethyl disulfide, N,N-dimethylformamide, dimethyl glutarate, 2,2-dimethylheptane, 2,2-dimethylhexane, 2,3-dimethylhexane, 2,4-dimethylhexane, 2,5-dimethylhexane, 3,3-dimethylhexane, 3,4-dimethylhexane, dimethyl maleate, 1,2-dimethylnaphthalene, 1,6-dimethylnaphthalene, 2,2-dimethylpentane, 2,3-dimethylpentane, 2,4-dimethylpentane, 3,3-dimethylpentane, dimethyl phthalate, 2,2-dimethyl-1-propanol, dimethyl succinate, 1,3-dioxolane, dipentene, dipentyl ether, diphenyl ether, dipropyl ether, dodecane, 1-dodecene, 1,2-epoxybutane, ethyl acetoacetate, ethyl acrylate, ethylbenzene, ethyl benzoate, ethyl butanoate, 2-ethyl-1-butanol, ethylbutyl ketone, ethyl trans-cinnamate, ethyl cyanoacetate, ethylcyclohexane, ethylene carbonate, ethylene glycol, ethylene glycol diacetate, ethylene glycol dibutyl ether, ethylene glycol diethyl ether, ethylene glycol dimethyl ether, ethylene glycol ethylether acetate, ethylene glycol monomethyl ether acetate, ethylene glycol monobutyl ether, ethylene glycol monoethylether, ethylene glycol monomethyl ether, 3-ethylhexane, 2-ethyl-1,3-hexanediol, 2-ethyl-1-hexanol, 2-ethylhexyl acetate, ethyl isovalerate, ethyl lactate, 3-ethyl-2-methylpentane, 3-ethyl-3-methylpentane, 3-ethylpentane, ethyl propanoate, fluorobenzene, o-, m-, p-fluorotoluene, formamide, furfuryl alcohol, glycerol, heptane, 1-heptanol, 2-heptanol, 3-heptanol, 1-heptene, cis,trans2-heptene, hexafluorobenzene, hexamethylphosphoric trimide, hexane, hexanenitrile, 1,2,6-hexanetriol, 1-hexanol, 2-hexanol, 3-hexanol, 1-hexene, cis,trans-2-hexene, cis,trans-3-hexene, hexyl acetate, sec-hexyl acetate, hexylene glycol, hexyl methyl ketone, hydraacrylonitrile, iodobenzene, 1-iodobutane, 2-iodobutane, iodoethane, 1-iodo-2-methylpropane, 1-iodopropane, 2-iodopropane, isobutyl acetate, isobutylbenzene, isobutyl formate, isobutyl isobutanoate, isopentyl acetate, isopentyl isopentanoate, isophorone, isopropyl acetate, D & L-limonene, 2,4-lutidine, 2,6-lutidine, mesitylene, mesityl oxide, n-methylacetamide, methyl acetate, methyl acetoacetate, methyl benzoate, 2-methyl-1-butanol, 3-methyl-1-butanol, 2-methyl-2-butanol, 3-methyl-2-butanol, methyl cyanoactate, methylcyclohexane, 1-methylcyclohexanol, cis,trans-2-methylcyclohexanol, cis,trans-3-methylcyclohexanol, cis,trans-4-methylcyclohexanol, methylcyclopentane, N-methylformamide, 2-methylheptane, 3-methylheptane, 4-methylheptane, 2-methylhexane, 3-methylhexane, methyl isobutyl ketone, methyl isopentyl ketone, 1-methylnaphthalene, 2-methyloctane, 3-methyloctane, 4-methyloctane, methyl oleate, 2-methylpentane, 3-methylpentane, 2-methyl-1-pentanol, 3-methyl-1-pentanol, 2-methyl-2-pentanol, 3-methyl-2-pentanol, 4-methyl-2-pentanol, 2-methyl-3-pentanol, 3-methyl-3-pentanol, 4-methyl-4-penten-2-one, methyl pentyl ketone, N-methylpropanamide, 2-methylpropanenitrile, 2-methyl-1-propanol, 2-methyl-2-propanol, methyl propyl ketone, N-methyl-2-pyrrolidone, methyl salicylate, 2-methyl tetrahydrofuran, 2-methylthiophene, 3-methylthiophene, 4-methylvaleronitrile, β-myracene, nitroethane, nitromethane, 1-nitropropane, 2-nitropropane, nonane, 1-nonene, octane, octanenitrile, 1-octanol, 2-octanol, 1-octene, cis,trans-2-octene, pentachloroethane, 1,5-pentanediol, pentanenitrile, 1-pentanol, 2-pentanol, 3-pentanol, pentyl acetate, β-phellandrene, phenetole, 2-picoline, 3-picoline, 4-picoline, α-pinene, β-pinene, 1,2-propanediol, 1,3-propanediol, propanenitrile, propargyl acetate, propargyl alcohol, propyl acetate, propylbenzene, propyl benzoate, propylene carbonate, propyl formate, pseudocumene, styrene, α-terpinene, terpinolene, 1,1,2,2-tetrabromoethane, 1,1,1,2-tetrachloroethane, 1,1,2,2-tetrachloroethane, tetrachloroethylene, tetrachloromethane, tetraethylene glycol, tetraethylsilane, tetrahydrofuran, tetrahydrofurfuryl alcohol, tetrahydronaphthalene, tetrahydropyran, tetrahydrothiophene, 2,2,3,3-tetramethylpentane, 2,2,3,4-tetramethylpentane, 2,2,4,4-tetramethylpentane, 2,3,3,4-tetramethylpentane, tetramethylurea, thiodiethanol, thiophene, toluene, o-, m-, p-toluidine, α-tolylnitrile, triacetin, tribromomethane, tributyl borate, tributyl phosphate, 1,1,1-trichloroethane, 1,1,2-trichloroethane, trichloroethylene, trichloroethylsilane, trichlorofluoromethane, (trichloromethyl)benzene, trichloromethylsilane, 1,2,3-trichloropropane, 1,1,2-trichlorotrifluoroethane, tri-o-cresyl phosphate, tridecane, 1-tridecene, triethylene glycol, triethyl phosphate, 2,2,2-trifluoroethanol, (trifluoromethyl)benzene, 1,2,3-trimethylbenzene, 2,2,3-trimethylbutane, 2,2,5-trimethylhexane, 2,3,5-trimethylhexane, 2,2,3-trimethylpentane, 2,2,4-trimethylpentane, 2,3,3-, trimethylpentane, 2,3,4-trimethylpentane, trimethyl phosphate, 1-undecene, veratrole, vinyl acetate o-, m-, p-xylene and combinations thereof.

8. The kit of claim 6 wherein the solvent is selected from the group consisting of water acetone, 2-butonone, 2-propanol, methanol, ethanol, 1-propanol, acetonitrile, ethyl acetate, 1-butanol, 2-butanol, n-butyl acetate, carbon tetrachloride, chlorobenzene, chloroform, cyclohexane, cyclopentane, o-, m-, p-dichlorobenzene, dimethyl acetamide, dimethyl sulfoxide, dioxane, 2-ethoxyethanol, ethylene dichloride, glyme, heptane, hexadecane, hexane, iso-hexanes, 2-methoxyethanol, methyl t-butyl ether, methyl isoamyl ketone, methyl n-propyl ketone, dichloromethane, N-methylpyrrolidine, nonane, pentane, petroleum ether, propylene carbonate, pyridine, tetrahydrofuran, toluene, benzene, trichloroethylene, 1,1,2-trichlorotrifluoroethane, 2,2,4-trimethylpentane, o-xylene, actal, acetamide, acetophenone, acetylacetone, adiponitrile, allyl acetate, allyl alcohol, anisole, benzenethiol, benzonitrile, benzyl acetate, benzyl alcohol, benzyl benzoate, benzyl chloride, benzyl ethyl ether, bis(2-chloroethyl)ether, bis(2-ethylhexyl acetate), bromobenzene, 1-bromobutane, 2-bromobutane, 1-bromo-2-chloroethane, bromochloromethane, 1-bromodecane, 2-bromo-2-methylproprane, 1-bromonaphthalene, 1-bromopentane, 1-bromopropane, 2-bromopropane, 1,3-butanediol, 1,4-butanediol, 2,3-butanediol, butanenitrile, butanethiol, cis & trans 2-butene-1,4-diol, butyl acetate, sec-butyl acetate, tert-butyl benzene, butyl ethyl ether, butyl formate, butyl methyl ketone, butyl stearate, p-tert-butyltoluene, butyl vinyl ether, γ-butyrolactone, 1-chloro-3-methylbutane, 3-(chloromethyl)heptane, 1-chloronaphthalene, 1-chlorooctane, 1-chloropentane, o-, m-, p-chlorotoluene, cineole, o-, m-, p-cresol, cis,trans-crotonyl alcohol, cumene, cyclohexaol, cyclohexanone, cyclohexene, cyclohexylbenzene, cyclopentanone, p-cymene, cis,trans-decahydronaphthalene, decane, 1-decene, diacetone alcohol, dibenzyl ether, 1,2-dibromo-1,1-difluoroetane, 1,2-dibromoethane, dibromofluoromethane, dibromomethane, 1,2-dibromopropane, dibutyl ether, dibutyl maleate, dibutyl phthalate, dibutyl sebacate, dibutyl sulfide, 1,2-dichloropropane, 2,4-dichlorotoluene, 3,4-dichlorotoluene, diethyl carbonate, diethylene glycol, diethylene glycol dibutyl ether, diethylene glycol, diethylene glycol diethyl ether, diethylene glycol dimethyl ether, dimethylene glycol monoethyl ether, diethylene glycol monoethyl ether acetate, diethylene glycol monomethyl ether, diethyl ketone, diethyl malonate, diethyl oxalate, 2,3-diethylpentane, diethylpentane, diethyl sulfide, diiodomethane, diisobutyl ketone, dipentyl ether, diisopropyl ether, diisopropyl ketone, dimethyl adipate, dimethyl aniline, 2,2-dimethylbutane, 2,3-dimethylbutane, 3,3-dimethyl-1-butanol, 2,3-dimethyl-2-butanol, 3,3-dimethyl-2-butanol, cis,trans-1,2-dimethylcyclohexane, dimethyl disulfide, N,N-dimethylformamide, dimethyl glutarate, 2,2-dimethylheptane, 2,2-dimethylhexane, 2,3-dimethylhexane, 2,4-dimethylhexane, 2,5-dimethylhexane, 3,3-dimethylhexane, 3,4-dimethylhexane, dimethyl maleate, 1,2-dimethylnaphthalene, 1,6-dimethylnaphthalene, 2,2-dimethylpentane, 2,3-dimethylpentane, 2,4-dimethylpentane, 3,3-dimethylpentane, dimethyl phthalate, 2,2-dimethyl-1-propanol, dimethyl succinate, 1,3-dioxolane, dipentene, dipentyl ether, diphenyl ether, dipropyl ether, dodecane, 1-dodecene, 1,2-epoxybutane, ethyl acetoacetate, ethyl acrylate, ethylbenzene, ethyl benzoate, ethyl butanoate, 2-ethyl-1-butanol, ethylbutyl ketone, ethyl trans-cinnamate, ethyl cyanoacetate, ethylcyclohexane, ethylene carbonate, ethylene glycol, ethylene glycol diacetate, ethylene glycol dibutyl ether, ethylene glycol diethyl ether, ethylene glycol dimethyl ether, ethylene glycol ethylether acetate, ethylene glycol monomethyl ether acetate, ethylene glycol monobutyl ether, ethylene glycol monoethylether, ethylene glycol monomethyl ether, 3-ethylhexane, 2-ethyl-1,3-hexanediol, 2-ethyl-1-hexanol, 2-ethylhexyl acetate, ethyl isovalerate, ethyl lactate, 3-ethyl-2-methylpentane, 3-ethyl-3-methylpentane, 3-ethylpentane, ethyl propanoate, fluorobenzene, o-, m-, p-fluorotoluene, formamide, furfuryl alcohol, glycerol, heptane, 1-heptanol, 2-heptanol, 3-heptanol, 1-heptene, cis,trans2-heptene, hexafluorobenzene, hexamethylphosphoric trimide, hexane, hexanenitrile, 1,2,6-hexanetriol, 1-hexanol, 2-hexanol, 3-hexanol, 1-hexene, cis,trans-2-hexene, cis,trans-3-hexene, hexyl acetate, sec-hexyl acetate, hexylene glycol, hexyl methyl ketone, hydraacrylonitrile, iodobenzene, 1-iodobutane, 2-iodobutane, iodoethane, 1-iodo-2-methylpropane, 1-iodopropane, 2-iodopropane, isobutyl acetate, isobutylbenzene, isobutyl formate, isobutyl isobutanoate, isopentyl acetate, isopentyl isopentanoate, isophorone, isopropyl acetate, D & L-limonene, 2,4-lutidine, 2,6-lutidine, mesitylene, mesityl oxide, n-methylacetamide, methyl acetate, methyl acetoacetate, methyl benzoate, 2-methyl-1-butanol, 3-methyl-1-butanol, 2-methyl-2-butanol, 3-methyl-2-butanol, methyl cyanoactate, methylcyclohexane, 1-methylcyclohexanol, cis,trans-2-methylcyclohexanol, cis,trans-3-methylcyclohexanol, cis,trans-4-methylcyclohexanol, methylcyclopentane, N-methylformamide, 2-methylheptane, 3-methylheptane, 4-methylheptane, 2-methylhexane, 3-methylhexane, methyl isobutyl ketone, methyl isopentyl ketone, 1-methylnaphthalene, 2-methyloctane, 3-methyloctane, 4-methyloctane, methyl oleate, 2-methylpentane, 3-methylpentane, 2-methyl-1-pentanol, 3-methyl-1-pentanol, 2-methyl-2-pentanol, 3-methyl-2-pentanol, 4-methyl-2-pentanol, 2-methyl-3-pentanol, 3-methyl-3-pentanol, 4-methyl-4-penten-2-one, methyl pentyl ketone, N-methylpropanamide, 2-methylpropanenitrile, 2-methyl-1-propanol, 2-methyl-2-propanol, methyl propyl ketone, N-methyl-2-pyrrolidone, methyl salicylate, 2-methyl tetrahydrofuran, 2-methylthiophene, 3-methylthiophene, 4-methylvaleronitrile, β-myracene, nitroethane, nitromethane, 1-nitropropane, 2-nitropropane, nonane, 1-nonene, octane, octanenitrile, 1-octanol, 2-octanol, 1-octene, cis,trans-2-octene, pentachloroethane, 1,5-pentanediol, pentanenitrile, 1-pentanol, 2-pentanol, 3-pentanol, pentyl acetate, β-phellandrene, phenetole, 2-picoline, 3-picoline, 4-picoline, α-pinene, β-pinene, 1,2-propanediol, 1,3-propanediol, propanenitrile, propargyl acetate, propargyl alcohol, propyl acetate, propylbenzene, propyl benzoate, propylene carbonate, propyl formate, pseudocumene, styrene, α-terpinene, terpinolene, 1,1,2,2-tetrabromoethane, 1,1,1,2-tetrachloroethane, 1,1,2,2-tetrachloroethane, tetrachloroethylene, tetrachloromethane, tetraethylene glycol, tetraethylsilane, tetrahydrofuran, tetrahydrofurfuryl alcohol, tetrahydronaphthalene, tetrahydropyran, tetrahydrothiophene, 2,2,3,3-tetramethylpentane, 2,2,3,4-tetramethylpentane, 2,2,4,4-tetramethylpentane, 2,3,3,4-tetramethylpentane, tetramethylurea, thiodiethanol, thiophene, toluene, o-, m-, p-toluidine, α-tolylnitrile, triacetin, tribromomethane, tributyl borate, tributyl phosphate, 1,1,1-trichloroethane, 1,1,2-trichloroethane, trichloroethylene, trichloroethylsilane, trichlorofluoromethane, (trichloromethyl)benzene, trichloromethylsilane, 1,2,3-trichloropropane, 1,1,2-trichlorotrifluoroethane, tri-o-cresyl phosphate, tridecane, 1-tridecene, triethylene glycol, triethyl phosphate, 2,2,2-trifluoroethanol, (trifluoromethyl)benzene, 1,2,3-trimethylbenzene, 2,2,3-trimethylbutane, 2,2,5-trimethylhexane, 2,3,5-trimethylhexane, 2,2,3-trimethylpentane, 2,2,4-trimethylpentane, 2,3,3-, trimethylpentane, 2,3,4-trimethylpentane, trimethyl phosphate, 1-undecene, veratrole, vinyl acetate o-, m-, p-xylene and combinations thereof.

9. A method for the rapid high throughput determination of the optimum solvents and resolving agents and conditions for the crystallization of diasteroisomeric salts to separate enantiomers, which method comprises:
A. Obtaining the kit of claim 1;
B. Adding to each container of the array of claim 1 a measured amount of racemic organic compound neat.
C. Heating the combination of sub-step B to a solubilization temperature not in excess of 100° C. for less than 15 minutes;
D. Optionally agitating the combination of substep C for between about 5 min. and 24 hr.;
E. Cooling the heated combination of sub-step C;
F. Observing the formation of diastereoisometric crystals visually or by optical means in each container;
G. Separating the formed diastereoisometric salts;
H. Isolating and evaluating the desired isomer; and
I. Selecting the optimal combinations of resolving agent and solvents and resolution conditions.

10. The method of claim 9 wherein the contents of each container are spherically agitated in substep D.

11. The method of claim 9 wherein agitation in substep D in each tube is obtained by use of a magnetic stirring bar or a shaking platform.

12. The method of claim 9 wherein the initial and final pressure and temperature are ambient conditions.

13. The method of claim 9 wherein the components in sub-steps C and D are heated to solubilization or to 100° C. and in sub-step E the components are allowed to cool to ambient temperature.

14. The method of claim 9 wherein after cooling the heated combination of sub-step F the temperature is at or lower than 20° C.

15. The method of claim 13 wherein the cooled combination is optionally cooled to 4° C. and then to 0° C.

16. The method of claim 9 wherein the resolution process described is repeated at least one time.

17. The method of claim 9 wherein the solvent in the kit in sub-step A is selected from the group consisting of 90% acetone, methyl ethyl ketone (2-butanone), i-butanol, 2-propanol, 90% 2-propanol, methanol, 80% methanol, ethanol, 96% ethanol, water, 1-propanol, 85% 1-propanol, acetonitrile, ethyl acetate, dichloromethane, chloroform, p-dioxane, methyl-t-butyl ether, toluene and tetrahydrofuran.

18. The method of claim 9 wherein the resolving agent, acid or base, in the kit in sub-step A is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)—BOC-aminocyclopent-4-ene-1-(S)-carboxylic acid, 3-(S)—BOC-aminocyclopent-4-ene-1-(R)-carboxylic acid, N-2-BOC-D-arginine hydrochloride, N-2-BOC-D-aspartic acid, N-2-BOC-3-(4-bipheny)alanine, N-2-BOC—N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N—BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N—BOC-1,2,3,4-tetrahydroisoquinoline-(3R)-carboxylic acid, N—BOC-1,2,3,4-tetrahydroisoquinoline-(3S)-carboxylic acid, N-2-BOC-3-(4′-thiazolyl)alanine, N—BOC-D-threonine, N-2-BOC—N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N—CBZ-D-alanine, N—CBZ-D-arginine, N—CBZ-D-aspartic acid, N—CBZ-O-tert-butyl-D-serine, CBZ-1-cyclohexylalanine, N—CBZ-D-glutamic acid, N—CBZ-D-histidine, N—CBZ-D-leucine, N—CBZ-1-tert-leucine DCHA salt, N—CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50% solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine, N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, bis {1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaninol, BOC-prolinol, N-butyl-2-amino-2-methyl-3-(3′,4′-dihydroxyphenyl)-propionate, CBZ-1-cyclohexylalaninol, N-2-CBZ-D-3-(1′naphthyl)alaninol, N-2-CBZ-D-3-(2′naphthyl)alaninol, N-1-phenylalaninol, 2-(2′-chlorobenzyl)benzyl-amine, 2-(3′-chlorobenzyl)benzyl-amine, 2-(4′-chlorobenzyl)benzylamine, (S)-cyclohexylalaninol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaninol, N-(3,4-dimethoxybenzyl)-1-phenylethylamine, 3,3-dimethyl-2-aminobutane, N,N-dimethyl-1-methylbenzylamine, N,N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl(R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaninol, (R)1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

19. The method of claim 9 wherein the solvent is selected from the group consisting of water acetone, 2-butonone, 2-propanol, methanol, ethanol, 1-propanol, acetonitrile, ethyl acetate, 1-butanol, 2-butanol, n-butyl acetate, carbon tetrachloride, chlorobenzene, chloroform, cyclohexane, cyclopentane, o-, m-, p-dichlorobenzene, dimethyl acetamide, dimethyl sulfoxide, dioxane, 2-ethoxyethanol, ethylene dichloride, glyme, heptane, hexadecane, hexane, iso-hexanes, 2-methoxyethanol, methyl t-butyl ether, methyl isoamyl ketone, methyl n-propyl ketone, dichloromethane, N-methylpyrrolidine, nonane, pentane, petroleum ether, propylene carbonate, pyridine, tetrahydrofuran, toluene, benzene, trichloroethylene, 1,1,2-trichlorotrifluoroethane, 2,2,4-trimethylpentane, o-xylene, actal, acetamide, acetophenone, acetylacetone, adiponitrile, allyl acetate, allyl alcohol, anisole, benzenethiol, benzonitrile, benzyl acetate, benzyl alcohol, benzyl benzoate, benzyl chloride, benzyl ethyl ether, bis(2-chloroethyl)ether, bis(2-ethylhexyl acetate), bromobenzene, 1-bromobutane, 2-bromobutane, 1-bromo-2-chloroethane, bromochloromethane, 1-bromodecane, 2-bromo-2-methylproprane, 1-bromonaphthalene, 1-bromopentane, 1-bromopropane, 2-bromopropane, 1,3-butanediol, 1,4-butanediol, 2,3-butanediol, butanenitrile, butanethiol, cis & trans 2-butene-1,4-diol, butyl acetate, sec-butyl acetate, tert-butyl benzene, butyl ethyl ether, butyl formate, butyl methyl ketone, butyl stearate, p-tert-butyltoluene, butyl vinyl ether, γ-butyrolactone, 1-chloro-3-methylbutane, 3-(chloromethyl)heptane, 1-chloronaphthalene, 1-chlorooctane, 1-chloropentane, o-, m-, p-chlorotoluene, cineole, o-, m-, p-cresol, cis,trans-crotonyl alcohol, cumene, cyclohexaol, cyclohexanone, cyclohexene, cyclohexylbenzene, cyclopentanone, p-cymene, cis,trans-decahydronaphthalene, decane, 1-decene, diacetone alcohol, dibenzyl ether, 1,2-dibromo-1,1-difluoroetane, 1,2-dibromoethane, dibromofluoromethane, dibromomethane, 1,2-dibromopropane, dibutyl ether, dibutyl maleate, dibutyl phthalate, dibutyl sebacate, dibutyl sulfide, 1,2-dichloropropane, 2,4-dichlorotoluene, 3,4-dichlorotoluene, diethyl carbonate, diethylene glycol, diethylene glycol dibutyl ether, diethylene glycol, diethylene glycol diethyl ether, diethylene glycol dimethyl ether, dimethylene glycol monoethyl ether, diethylene glycol monoethyl ether acetate, diethylene glycol monomethyl ether, diethyl ketone, diethyl malonate, diethyl oxalate, 2,3-diethylpentane, diethylpentane, diethyl sulfide, diiodomethane, diisobutyl ketone, dipentyl ether, diisopropyl ether, diisoprpyl ketone, dimethyl adipate, dimethyl aniline, 2,2-dimethylbutane, 2,3-dimethylbutane, 3,3-dimethyl-1-butanol, 2,3-dimethyl-2-butanol, 3,3-dimethyl-2-butanol, cis,trans-1,2-dimethylcyclohexane, dimethyl disulfide, N,N-dimethylformamide, dimethyl glutarate, 2,2-dimethylheptane, 2,2-dimethylhexane, 2,3-dimethylhexane, 2,4-dimethylhexane, 2,5-dimethylhexane, 3,3-dimethylhexane, 3,4-dimethylhexane, dimethyl maleate, 1,2-dimethylnaphthalene, 1,6-dimethylnaphthalene, 2,2-dimethylpentane, 2,3-dimethylpentane, 2,4-dimethylpentane, 3,3-dimethylpentane, dimethyl phthalate, 2,2-dimethyl-1-propanol, dimethyl succinate, 1,3-dioxolane, dipentene, dipentyl ether, diphenyl ether, dipropyl ether, dodecane, 1-dodecene, 1,2-epoxybutane, ethyl acetoacetate, ethyl acrylate, ethylbenzene, ethyl benzoate, ethyl butanoate, 2-ethyl-1-butanol, ethylbutyl ketone, ethyl trans-cinnamate, ethyl cyanoacetate, ethylcyclohexane, ethylene carbonate, ethylene glycol, ethylene glycol diacetate, ethylene glycol dibutyl ether, ethylene glycol diethyl ether, ethylene glycol dimethyl ether, ethylene glycol ethylether acetate, ethylene glycol monomethyl ether acetate, ethylene glycol monobutyl ether, ethylene glycol monoethylether, ethylene glycol monomethyl ether, 3-ethylhexane, 2-ethyl-1,3-hexanediol, 2-ethyl-1-hexanol, 2-ethylhexyl acetate, ethyl isovalerate, ethyl lactate, 3-ethyl-2-methylpentane, 3-ethyl-3-methylpentane, 3-ethylpentane, ethyl propanoate, fluorobenzene, o-, m-, p-fluorotoluene, formamide, furfuryl alcohol, glycerol, heptane, 1-heptanol, 2-heptanol, 3-heptanol, 1-heptene, cis,trans2-heptene, hexafluorobenzene, hexamethylphosphoric trimide, hexane, hexanenitrile, 1,2,6-hexanetriol, 1-hexanol, 2-hexanol, 3-hexanol, 1-hexene, cis,trans-2-hexene, cis,trans-3-hexene, hexyl acetate, sec-hexyl acetate, hexylene glycol, hexyl methyl ketone, hydraacrylonitrile, iodobenzene, 1-iodobutane, 2-iodobutane, iodoethane, 1-iodo-2-methylpropane, 1-iodopropane, 2-iodopropane, isobutyl acetate, isobutylbenzene, isobutyl formate, isobutyl isobutanoate, isopentyl acetate, isopentyl isopentanoate, isophorone, isopropyl acetate, D & L-limonene, 2,4-lutidine, 2,6-lutidine, mesitylene, mesityl oxide, n-methylacetamide, methyl acetate, methyl acetoacetate, methyl benzoate, 2-methyl-1-butanol, 3-methyl-1-butanol, 2-methyl-2-butanol, 3-methyl-2-butanol, methyl cyanoactate, methylcyclohexane, 1-methylcyclohexanol, cis,trans-2-methylcyclohexanol, cis,trans-3-methylcyclohexanol, cis,trans-4-methylcyclohexanol, methylcyclopentane, N-methylformamide, 2-methylheptane, 3-methylheptane, 4-methylheptane, 2-methylhexane, 3-methylhexane, methyl isobutyl ketone, methyl isopentyl ketone, 1-methylnaphthalene, 2-methyloctane, 3-methyloctane, 4-methyloctane, methyl oleate, 2-methylpentane, 3-methylpentane, 2-methyl-1-pentanol, 3-methyl-1-pentanol, 2-methyl-2-pentanol, 3-methyl-2-pentanol, 4-methyl-2-pentanol, 2-methyl-3-pentanol, 3-methyl-3-pentanol, 4-methyl-4-penten-2-one, methyl pentyl ketone, N-methylpropanamide, 2-methylpropanenitrile, 2-methyl-1-propanol, 2-methyl-2-propanol, methyl propyl ketone, N-methyl-2-pyrrolidone, methyl salicylate, 2-methyl tetrahydrofuran, 2-methylthiophene, 3-methylthiophene, 4-methylvaleronitrile, β-myracene, nitroethane, nitromethane, 1-nitropropane, 2-nitropropane, nonane, 1-nonene, octane, octanenitrile, 1-octanol, 2-octanol, 1-octene, cis,trans-2-octene, pentachloroethane, 1,5-pentanediol, pentanenitrile, 1-pentanol, 2-pentanol, 3-pentanol, pentyl acetate, 0-phellandrene, phenetole, 2-picoline, 3-picoline, 4-picoline, α-pinene, ⊖-pinene, 1,2-propanediol, 1,3-propanediol, propanenitrile, propargyl acetate, propargyl alcohol, propyl acetate, propylbenzene, propyl benzoate, propylene carbonate, propyl formate, pseudocumene, styrene, α-terpinene, terpinolene, 1,1,2,2-tetrabromoethane, 1,1,1,2-tetrachloroethane, 1,1,2,2-tetrachloroethane, tetrachloroethylene, tetrachloromethane, tetraethylene glycol, tetraethylsilane, tetrahydrofuran, tetrahydrofurfuryl alcohol, tetrahydronaphthalene, tetrahydropyran, tetrahydrothiophene, 2,2,3,3-tetramethylpentane, 2,2,3,4-tetramethylpentane, 2,2,4,4-tetramethylpentane, 2,3,3,4-tetramethylpentane, tetramethylurea, thiodiethanol, thiophene, toluene, o-, m-, p-toluidine, α-tolylnitrile, triacetin, tribromomethane, tributyl borate, tributyl phosphate, 1,1,1-trichloroethane, 1,1,2-trichloroethane, trichloroethylene, trichloroethylsilane, trichlorofluoromethane, (trichloromethyl)benzene, trichloromethylsilane, 1,2,3-trichloropropane, 1,1,2-trichlorotrifluoroethane, tri-o-cresyl phosphate, tridecane, 1-tridecene, triethylene glycol, triethyl phosphate, 2,2,2-trifluoroethanol, (trifluoromethyl)benzene, 1,2,3-trimethylbenzene, 2,2,3-trimethylbutane, 2,2,5-trimethylhexane, 2,3,5-trimethylhexane, 2,2,3-trimethylpentane, 2,2,4-trimethylpentane, 2,3,3-, trimethylpentane, 2,3,4-trimethylpentane, trimethyl phosphate, 1-undecene, veratrole, vinyl acetate o-, m-, p-xylene and combinations thereof.

20. The method of claim 19 wherein the resolving agent in the kit in sub-step A is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-argine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)—BOC-aminocyclopent-4-ene-1-(S)-carboxylic acid, 3-(S)—BOC-aminocyclopent-4-ene-1-(R)-carboxylic acid, N-2-BOC-D-arginine hydrochloride, N-2-BOC-D-aspartic acid, N-2-BOC-3-(4-bipheny)alanine, N-2-BOC—N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-Naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N—BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N—BOC-1,2,3,4-tetrahydroisoquinoline-(3R)-carboxylic acid, N—BOC-1,2,3,4-tetrahydroisoquinoline-(3S)-carboxylic acid, N-2-BOC-3-(4′-thiazolyl)alanine, N—BOC-D-threonine, N-2-BOC—N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2r,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N—CBZ-D-alanine, N—CBZ-D-arginine, N—CBZ-D-aspartic acid, N—CBZ-D-tert-butyl-D-serine, CBZ-1-cyclohexylalanine, N—CBZ-D-glutamic acid, N—CBZ-D-histidine, N—CBZ-D-leucine, N—CBZ-1-tert-leucine DCHA salt, N—CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50% solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine, N-methylglucamine (−), α-methylbenzylamine, cinochonidine (−), ephedrine (−), hydroquinidine (+), n-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindaN-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, Bis(methoxymethyl)pyrrolidine, Bis {1-[1-naphthyl]ethyl}amine hydrochloride, Bis(1-phenylethyl)amine hydrochloride, N,N-bis-[1-phenylethyl]phthalamic acid, N-2-BOC-cyclohexylglycine, BOC-isoleucinol, BOC-phenylalaninol, BOC-prolinol, N-butyl-2-amino-2-methyl-3-(3′,4′-dihydroxyphenyl)-propionate, CBZ-1-cyclohexylalaninol, N-2-CBZ-D-3-(1′naphthyl)alaninol, N-2-CBZ-D-3-(2′naphthyl)alaninol, N-1-phenylalaninol, 2-(2′-chlorobenzyl)benzyl-amine, 2-(3′-chlorobenzyl)benzyl-amine, 2-(4′-chlorobenzyl)benzylamine, (S)-cyclohexylalaninol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaninol, N-(3,4-dimethoxybenzyl)-1-phenylethylamine, 3,3-dimethyl-2-aminobutane, N,N-dimethyl-1-methylbenzylamine, N,N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl(R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin derivative, 6-methoxy-2-aminotetralin and N-propyl derivative, 7-methoxy-2-aminotetralin and N-propyl, 8-methoxy-2-aminotetralin and N-propyl derivative, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaninol, (R)1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

21. The kit of claim 1 wherein each substantially identical container has an attached unique marking to facilitate identification.

22. The kit of claim 1 wherein the unique marking is a bar code, an alphanumeric code, or combinations thereof.

23. The kit of claim 1 wherein the containers, rack, sealant and stopper are all stable at temperatures between with −20° C. to 120° C. and inert to the racemate, solvent and resolving agent.
